(19) United States
(12) Patent Application Publication
(10) Pub. No.: US 2005/0165085 A1
(43) Pub. Date: Jul. 28, 2005

(54) Polymorphic forms of tegaserod base and salts thereof

(76) Inventors: Marioara Mendelovici, Rechovot (IL); Judith Aronhime, Rehovot (IL); Santiago Ini, Haifa (IL); Greta Sterimbaum, Rishon-Lezion (IL); Eran Luvchick, Azor (IL)

Correspondence Address: KENYON & KENYON, ONE BROADWAY, NEW YORK, NY, 10004, US

(21) Appl. No.: 11/015,875

(22) Filed: Dec. 16, 2004

Related U.S. Application Data
(60) Provisional application No. 60/530,278, filed on Dec. 16, 2003.
(60) Provisional application No. 60/585,423, filed on Jul. 2, 2004.
(60) Provisional application No. 60/609,715, filed on Sep. 14, 2004.

(51) Int. Cl. A61K 31/405; C07D 209/14

(52) U.S. Cl. 514/419; 548/505

(57) ABSTRACT

Provided are polymorphic forms of tegaserod base and maleate, and processes for their preparation.

### CROSS-REFERENCE TO RELATED APPLICATIONS

This application claims the benefit of U.S. Provisional Application Nos. 60/530,278 filed on Dec. 16, 2003, 60/585,423 filed on Jul. 2 2004, and 60/609,715 filed on Sep. 14, 2004, the disclosure of which are incorporated by reference in its entirety herein.

### FIELD OF THE INVENTION

The present invention relates to solid state chemistry of tegaserod base and salts thereof.

### BACKGROUND OF THE INVENTION

Tegaserod maleate is an aminoguanidine indole 5HT4 agonist for the treatment of irritable bowel syndrome (IBS). Tegaserod maleate has the following structure: 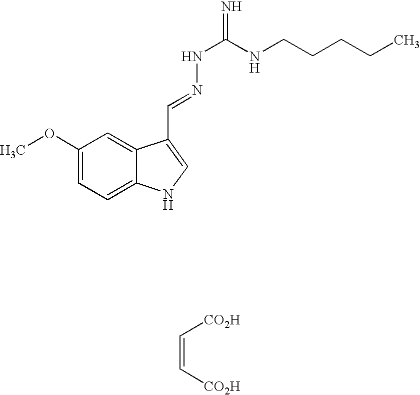
According to the prescribing information (Physician's Desk Reference, 57^{th Ed., at Page} 2339), tegaserod as the maleate salt is a white to off-white crystalline powder and is slightly soluble in ethanol and very slightly soluble in water. IPCOM000021161D characterizes the marketed polymorphic form of tegaserod maleate (ZELNORM), and designates the crystalline form of ZELNORM as tegaserod maleate Form A, which is characterized by an X-ray Diffraction pattern with peaks at 5.4, 6.0, 6.6 and 10.8±0.2 degrees two theta. The crystalline form is further characterized by an X-ray Diffraction pattern having peaks at about 5.9, 6.4, 11.5, 12.0, 14.8, 15.4, 16.2, 18.1, 19.4, 21.7, 23.9, 26.8 and 29.7±0.2 degrees two theta.

One embodiment of the present invention relates to the solid state physical properties of tegaserod base and salts thereof. These properties may be influenced by controlling the conditions under which tegaserod base or its salt is obtained in solid Form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid may have therapeutic consequences since it imposes an upper limit on the rate at which an orally-administered active ingredient may reach the patient's bloodstream. The rate of dissolution is also a consideration in formulating syrups, elixirs and other liquid medicaments. The solid state Form of a compound may also affect its behavior on compaction and its storage stability.

These practical physical characteristics are influenced by the conformation and orientation of molecules in the unit cell, which defines a particular polymorphic Form of a substance. The polymorphic form may give rise to thermal behavior different from that of the amorphous material or another polymorphic Form. Thermal behavior is measured in the laboratory by such techniques as capillary melting point, thermogravimetric analysis (TGA) and differential scanning calorimetry (DSC) and may be used to distinguish some polymorphic forms from others. A particular polymorphic Form may also give rise to distinct spectroscopic properties that may be detectable by powder X-ray crystallography, solid state C NMR spectrometry and infrared spectrometry.

Tegaserod maleate is disclosed in U.S. Pat. No. 5,510,353 and in its equivalent EP 0 505 322 (example 13). The '353 patent also discloses the preparation of tegaserod base by reacting indole-3-carbaldehyde and aminoguanidine in a protic solvent in the presence of inorganic or organic acid (example 2a describes the reaction in methanol and hydrochloric acid). The '353 patent however provides no detailed procedure to crystallize the base. Moreover the procedure to obtain the crystalline maleate salt from the base is completely absent. Tegaserod base and tegaserod maleate are chacterized in the '353 patent by a melting point of 124 and 190° C. respectively (table 1 example 13).

The literature (Buchheit K. H, et al., J. Med. Chem., 1995, 38, 2331) describes a general method for the condensation of aminoguanidines with indole-3-carbadehydes in methanol in the presence of HCl (pH 3-4). The product obtained after solvent evaporation may be converted to its hydrochloride salt by treatment of the methanolic solution with diethylether/HCl followed by recrystallization from methanol/diethylether. Tegaserod base prepared according to this general method is characterized solely by a melting point of 155° C. (table 3 compound 5b). Additional Tegaserod maleate characterization was done by ¹H and ¹³C-NMR according to the literature (Jing J. et. al., Guangdong Weiliang Yuansu Kexue, 2002, 9/2, 51).

Chinese patent No. CN 1425651 A, presents X-ray diffractograms of two crystalline forms. Forms B2 and C which are characterized by the present applicants match the X-Ray powder diffraction of Chinese patent No. CN 142565 1A. Form S of CN 1425651A is however defined as a hydrate and may have a different molecular composition at least in respect to Form B2, discussed in further detail below.

WO 04/085393 discloses four crystalline forms of tegaserod maleate. The search report for WO 04/085393 further identifies WO 00/10526, and Drugs Fut. 1999, 24(1) which provides an overview for tegaserod maleate.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

In addition to allowing for improved formulations, the polymorphic forms may be used for calibration of XRD, FTIR or DSC instruments. The polymorphic forms may further help in purification of tegaserod, particularly if they possess high crystallinity. In the event of metastability, a metastable polymorphic form may be used to prepare a more stable polymorph. Hence, discovery of new polymorphic forms and new processes help in advancing a formulation scientist in preparation of tegaserod as an active pharmaceutical ingredient in a formulation.

The present invention provides additional polymorphic forms of tegaserod and salts thereof.

### SUMMARY OF THE INVENTION

In one aspect, the present invention provides a process for preparing crystalline form of tegaserod maleate characterized by an X-ray Diffraction pattern having peaks at 5.4, 6.0, 6.6 and 10.8±0.2 degrees two theta (Form A) comprising:
a) preparing a solution of tegaserod maleate in an solvent; and
b) recovering the crystalline form as a precipitate.
   wherein the solvent is selected from the group consisting of acetonitrile, butyl lactate, methyl ethyl ketone, butanol, dioxane, ethanol, isopropanol, chloroform, ethoxyethanol, 2-ethoxyethanol, pyrolidone, dimethyl sulfoxide, N,N-Dimethylformamide, 1-methyl-2-pyrrolidone, N,N-Dimethylacetamide, water and mixtures thereof, with the proviso that water is not used as an individual solvent.

In another aspect, the present invention provides a process for preparing crystalline tegaserod maleate characterized by an X-ray Diffraction pattern having peaks at 5.4, 6.0, 6.6 and 10.8±0.2 degrees two theta (Form A) comprising heating a solvate of tegaserod maleate to cause desolvation.

In another aspect, the present invention provides a process for preparing crystalline form of tegaserod maleate characterized by an X-ray Diffraction pattern having peaks at 5.4, 6.0, 6.6 and 10.8±0.2 degrees two theta (Form A) comprising:
a) combining a solution of maleic acid in a solvent with a solution of tegaserod free base in the same or different solvent; and
b) recovering the crystalline form as a precipitate.
   wherein the solvent is selected from the group consisting of acetonitrile, n-butanol, dioxane, methyl ethyl ketone, ethyl lactate, ethyl acetate and water.

In another aspect, the present invention provides a process crystalline form of tegaserod maleate (Form B) characterized by an X-ray Diffraction pattern having peaks at 15.7, 16.9, 17.2, 24.1, 24.6 and 25.2±0.2 two theta.

In another aspect, the present invention provides a process for preparing Form B comprising slurrying a tegaserod maleate in solid state in 1-propanol, and recovering the crystalline form.

In another aspect, the present invention provides a crystalline form of tegaserod maleate (Form B1) characterized by an X-ray Diffraction pattern having peaks at 10.3, 16.1, 16.5, 17.1, 20.3, 22.0, and 25.3±0.2 two theta.

In another aspect, the present invention provides a process for preparing crystalline Form B1 comprising:
a) preparing a solution of tegaserod maleate in chloroform, optionally in mixture with methanol or ethanol; and
b) recovering the crystalline form as a precipitate.

In another aspect, the present invention provides a crystalline tegaserod maleate characterized by an X-ray Diffraction pattern having peaks at 8.7, 15.6, 16.0, 22.2, 25.3 and ±0.2 two theta (Form B2), wherein the crystalline form is an ethanolate solvate.

In another aspect, the present invention provides a process for preparing the crystalline Form B2 comprising:
a) slurrying a crystalline form of tegaserod maleate in ethanol; and
b) recovering the crystalline tegaserod maleate.

In another aspect, the present invention provides a crystalline form of tegaserod maleate (Form B3) characterized by an X-ray Diffraction pattern having peaks at 15.6, 16.0, 22.5, 25.5 and 29.3±0.2 two theta.

In another aspect, the present invention provides a process for preparing Form B3 comprising crystallizing the crystalline form from ethanol, or slurrying tegaserod maleate in ethanol or contacting tegaserod maleate with vapors of ethanol.

In another aspect, the present invention provides a process for preparing Form B3 comprising:
a) combining a solution of maleic acid in ethanol with a solution of tegaserod free base in ethanol; and
b) recovering the crystalline form as a precipitate.

In another aspect, the present invention provides a process for preparing crystalline tegaserod maleate characterized by an X-ray Diffraction pattern having peaks at 7.8, 8.7, 17.1, 17.3 and 25.1±0.2 two theta (Form C) comprising heating crystalline tegaserod maleate characterized by an XRD pattern with peaks at 8.7, 15.6, 16.0, 22.2, 25.3 and ±0.2 two theta (Form B2) at a temperature of at least about 40° C.

In another aspect, the present invention provides a crystalline form of tegaserod maleate (Form D) having an X-ray powder diffraction with peaks at about 14.6, 20.2, 23.8, 26.0, 28.6 and 29.3±0.2 two theta.

In another aspect, the present invention provides a process for preparing the crystalline Form D, comprising slurrying or crystallizing the crystalline form in a solvent selected from the group consisting of 1-methyl-2-pyrrolidone, n-propanol and mixtures thereof.

In another Aspect, the present invention provides a crystalline form of tegaserod maleate (Form E) having an X-ray powder diffraction with peaks at 10.3, 16.6, 17.1, 22.0 and 25.4±0.2 two theta.

In another aspect, the present invention provides a process for preparing crystalline Form E comprising:
a) slurrying tegaserod maleate in dioxane; and
b) recovering the crystalline form.

In another aspect, the present invention provides a process for preparing the crystalline Form E comprising:
a) combining a solution of maleic acid in tetrahydrofuran with a solution of tegaserod free base in tetrahydrofuran; and
b) recovering the crystalline form as a precipitate.

In another aspect, the present invention provides a crystalline form of tegaserod hemi-maleate having an X-ray powder diffraction with peaks at 5.0, 9.9, 19.8, and 25.9±0.2 two theta.

In another aspect, the present invention provides a process for preparing crystalline tegaserod hemi-maleate comprising:
a) combining tegaserod base, maleic acid and ethyl acetate to obtain a reaction mixture;
b) heating the reaction mixture; and
c) recovering the crystalline form as a precipitate.

In another aspect, the present invention provides a crystalline form of tegaserod base (Form F) having an X-ray powder diffraction with peaks at 10.2, 11.3, 20.3, 21.3, 21.8, 27.6, 29.6, 31.1 and 32.7±0.2 two theta.

In another aspect, the present invention provides a process for preparing the crystalline Form F, comprising:
a) preparing a solution of tegaserod in a C₁ to C₈ chlorinated aliphatic hydrocarbon; and
b) removing the chlorinated hydrocarbon.

In another aspect, the present invention provides a crystalline form of tegaserod base (Form H) having an X-ray powder diffraction with peaks at 8.8, 15.1, 17.6, 21.8 and 23.9±0.2 two theta.

In another aspect, the present invention provides a process for preparing crystalline Form H comprising:
a) preparing a solution of tegaserod base in ethanol; and
b) recovering the crystalline form as a precipitate.

In another aspect, the present invention provides a process for preparing Form H comprising:
a) slurrying tegaserod base in ethyl acetate; and
b) recovering the crystalline form from the slurry.

In another aspect, the present invention provides amorphous tegaserod base in the solid state.

In another aspect, the present invention provides a process for preparing amorphous tegaserod comprising:
a) preparing a solution of tegaserod in an organic solvent; and
b) removing the solvent.

In another aspect, the present invention provides tegaserod acetate in solid state.

In another aspect, the present invention provides a crystalline tegaserod acetate.

In another aspect, the present invention provides a crystalline form of tegaserod acetate (Form J) having an X-ray powder diffraction with peaks at about 7.3, 8.7, 10.9 and 13.5±0.2 two theta.

In another aspect, the present invention provides a process for preparing Form J comprising:
a) combining tegaserod maleate (or other salt or free base), ethyl acetate or acetic acid, and a base under aqueous condition to obtain a reaction mixture; and
b) recovering the crystalline form.

In another aspect, the present invention provides a process for preparing Form J comprising:
a) slurrying tegaserod base amorphous in ethyl acetate; and
b) recovering the crystalline form.

In another aspect, the present invention provides a pharmaceutical composition comprising a polymorphic form of tegaserod base, maleate or acetate selected from the group consisting of B, B1, B3, D, E, J, tegaserod hemimaleate and a pharmaceutically acceptable excipient; and method of treating a huarm suffering from irritable bowel syndrome comprising administering the composition to the human in need thereof.

In another tspect, the present invention provides a solvate of tegaserod maleate, wherein the maleate is a solvate of a solvent selected from the group consisting of ethanol, isopropanol, 1-propanol, chloroform and dioxane.

In another aspect, the present invention provides a pharmaceutical composition comprising a polymorphic form of tegaserod base or maleate selected from the group consisting of B, B1, B3, D, E, J, tegaserod hemimaleate, for use in treatment of irritable bowl syndrome.

### BRIEF DESCRIPTION OF THE FIGURES

FIG. 1 is an X-Ray powder diffraction of tegaserod maleate Form A.

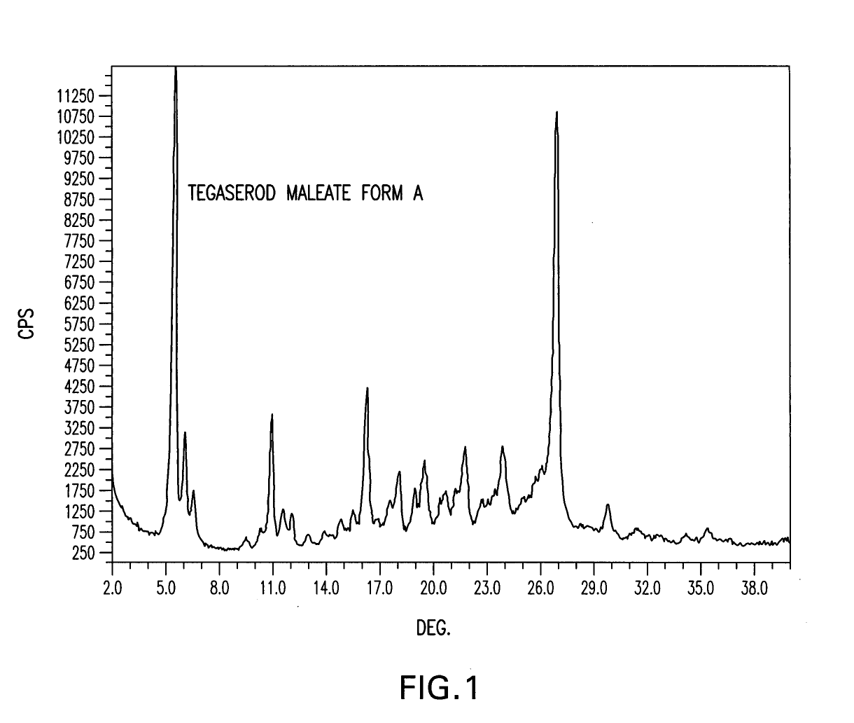

FIG. 2 is an X-Ray powder diffraction of tegaserod maleate Form B.

FIG. 3 is an X-Ray powder diffraction of tegaserod maleate Form B1.

FIG. 4 is an X-Ray powder diffraction of tegaserod maleate Form B2.

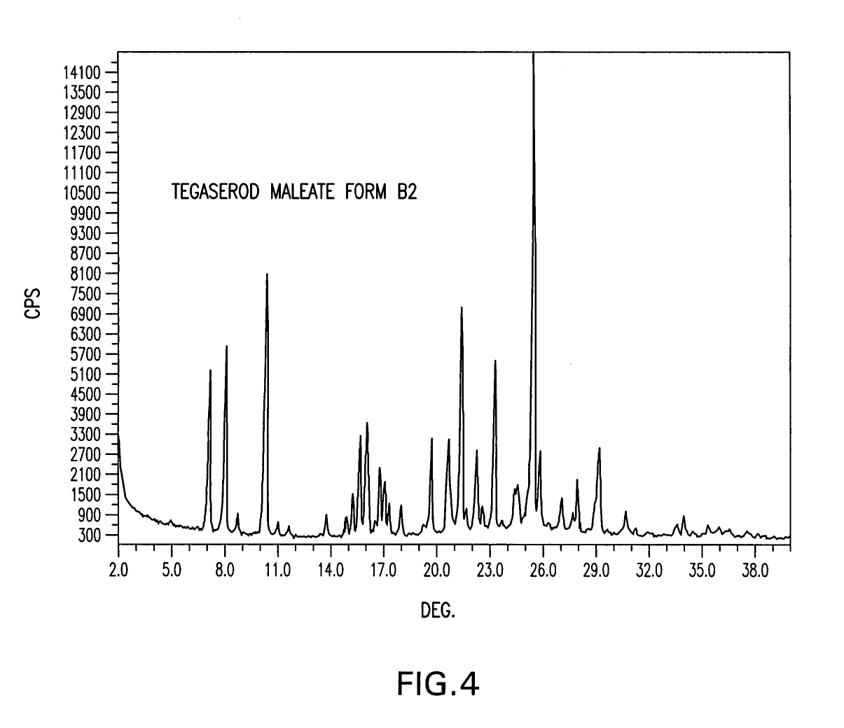

FIG. 5 is an X-Ray powder diffraction of tegaserod maleate Form B3.

FIG. 6 is an X-Ray powder diffraction of tegaserod maleate Form C.

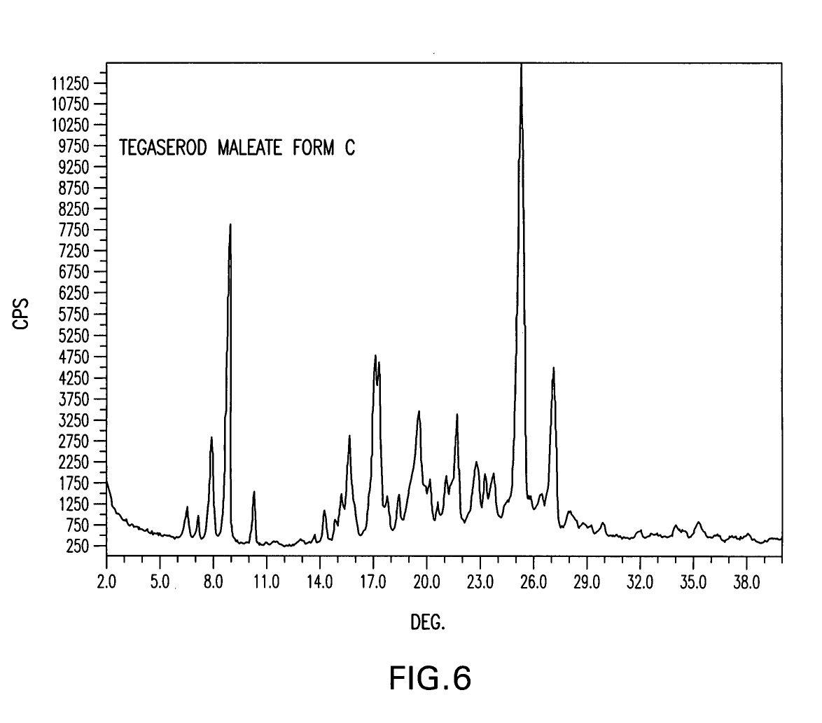

FIG. 7 is an X-Ray powder diffraction of tegaserod maleate Form D.

FIG. 8 is an X-Ray powder diffraction of tegaserod maleate Form E.

FIG. 9 is an X-Ray powder diffraction of tegaserod base Form F.

FIG. 10 is an X-Ray powder diffraction of tegaserod base Form H.

FIG. 11 is an X-Ray powder diffraction of tegaserod base amorphous.

FIG. 12 is an X-Ray powder diffraction of tegaserod acetate Form J.

FIG. 13 is an X-Ray powder diffraction of tegaserod hemi-maleate.

FIG. 14 is a DSC curve of tegaserod maleate Form A.

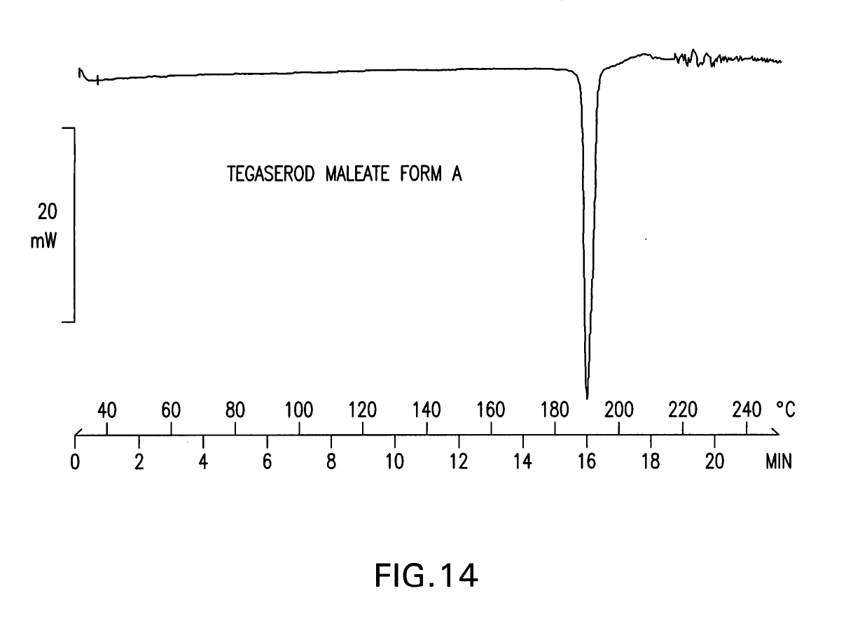

FIG. 15 is a DSC curve of tegaserod maleate Form B.

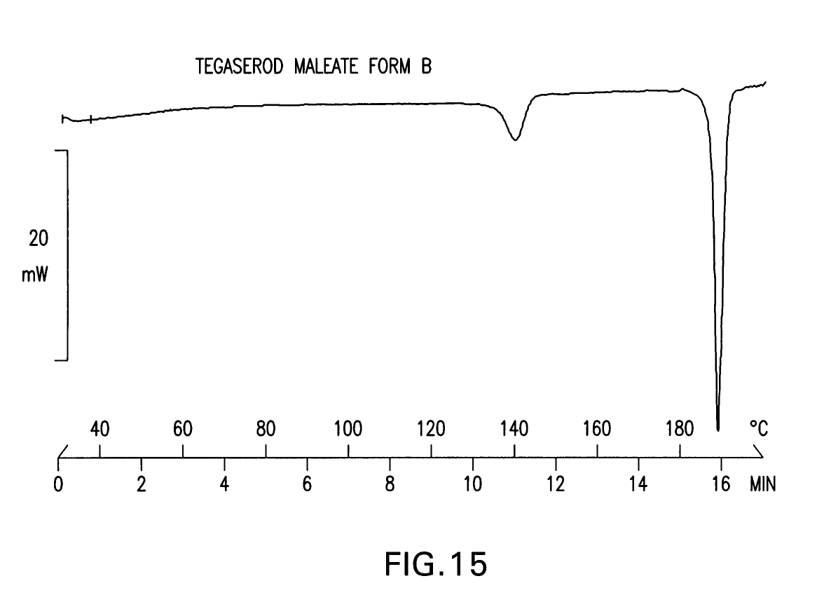

FIG. 16 is a DSC curve of tegaserod maleate Form B1.

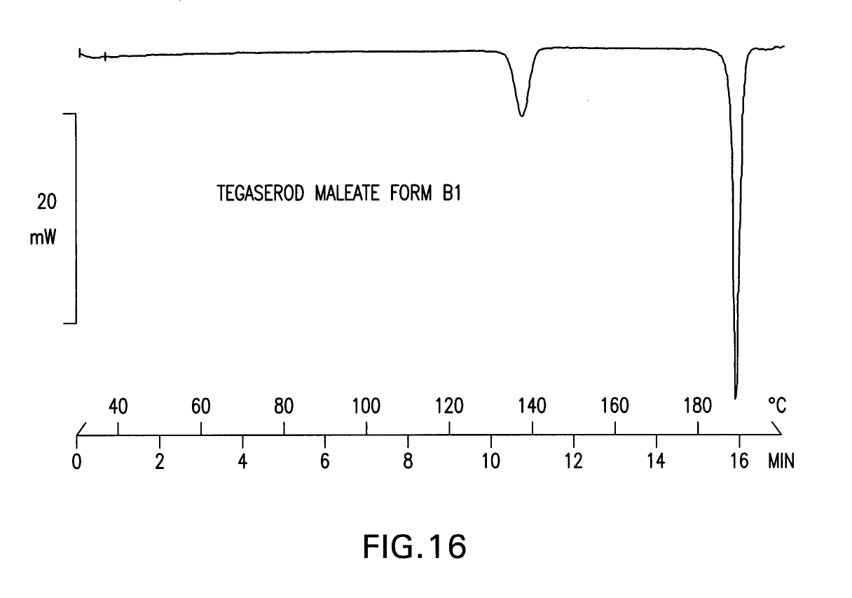

FIG. 17 is a DSC curve of tegaserod maleate Form B2.

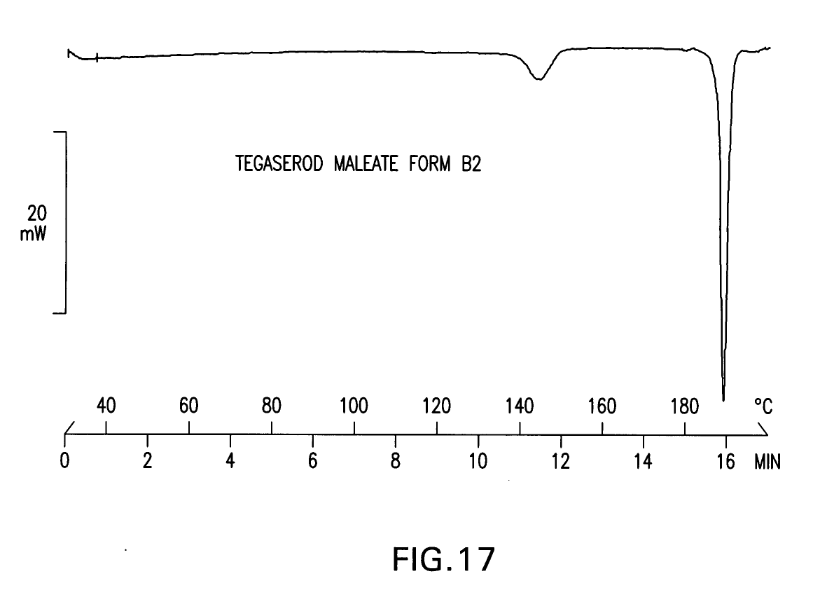

FIG. 18 is a DSC curve of tegaserod maleate Form B3.

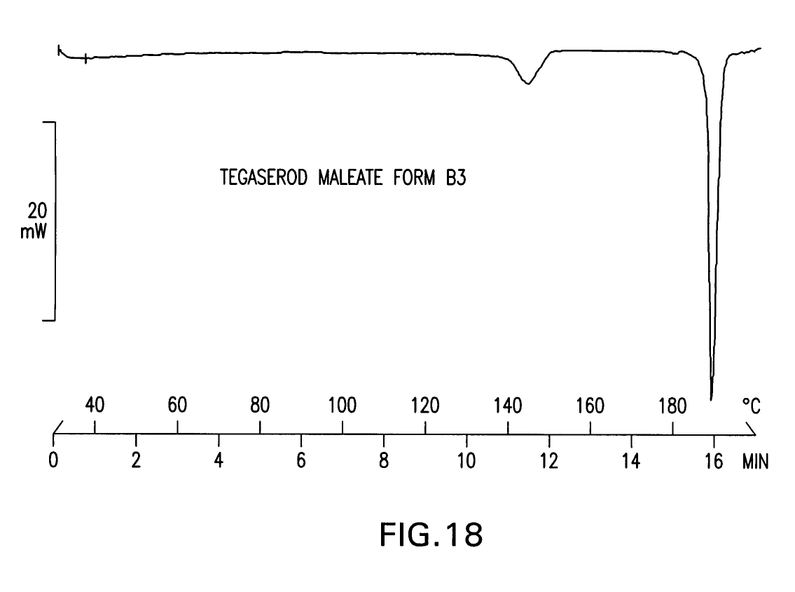

FIG. 19 is a DSC curve of tegaserod maleate Form C.

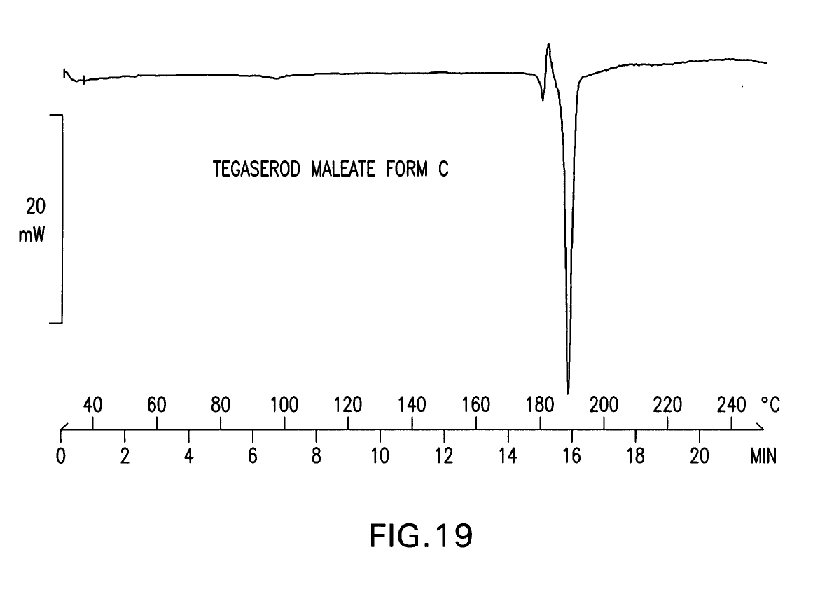

FIG. 20 is a DSC curve of tegaserod maleate Form E.

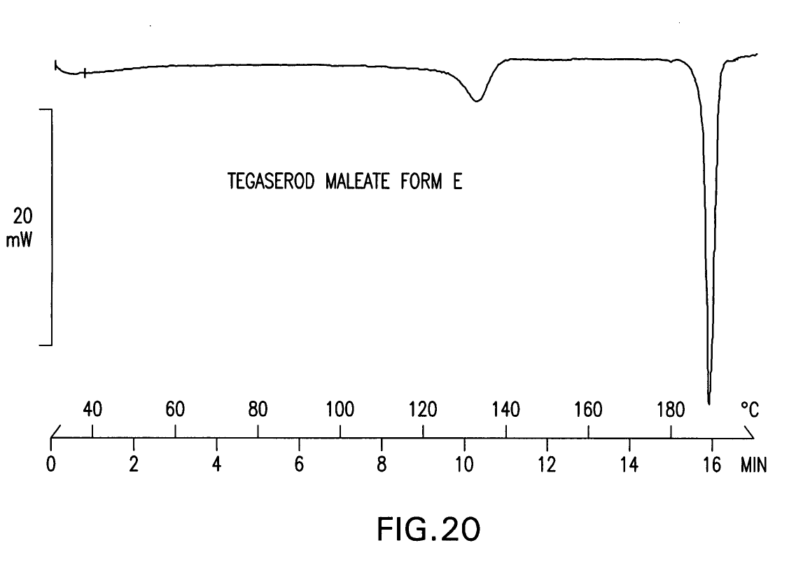

FIG. 21 is a DSC curve of tegaserod maleate Form F.

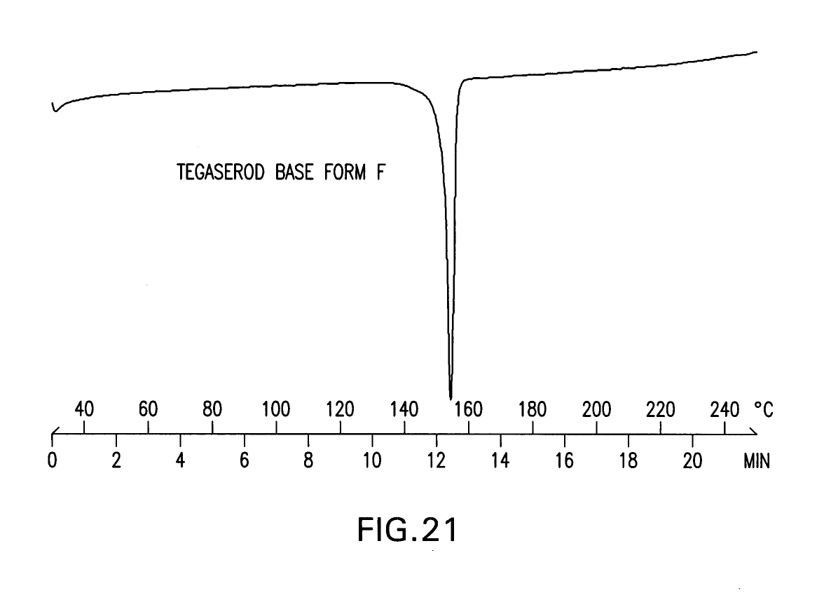

FIG. 22 is a DSC curve of tegaserod base Form H.

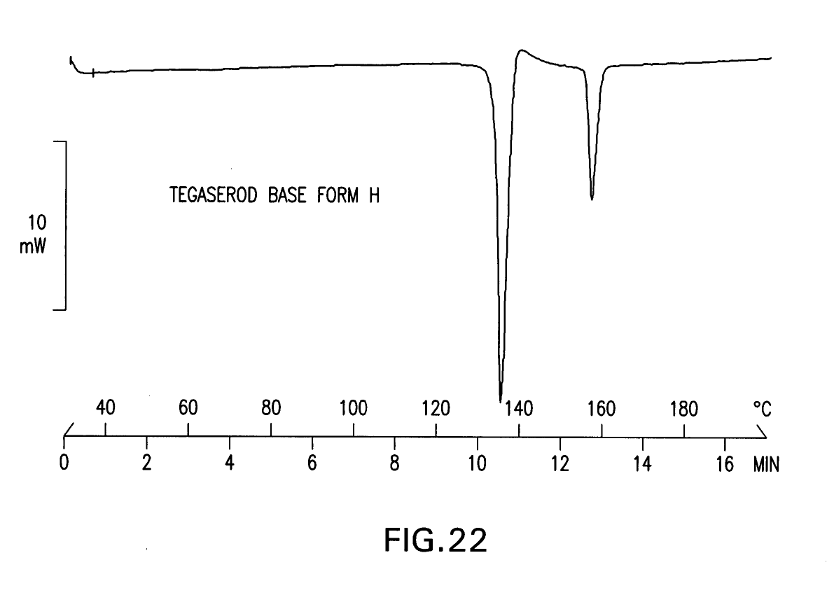

FIG. 23 is a DSC curve of tegaserod base amorphous.

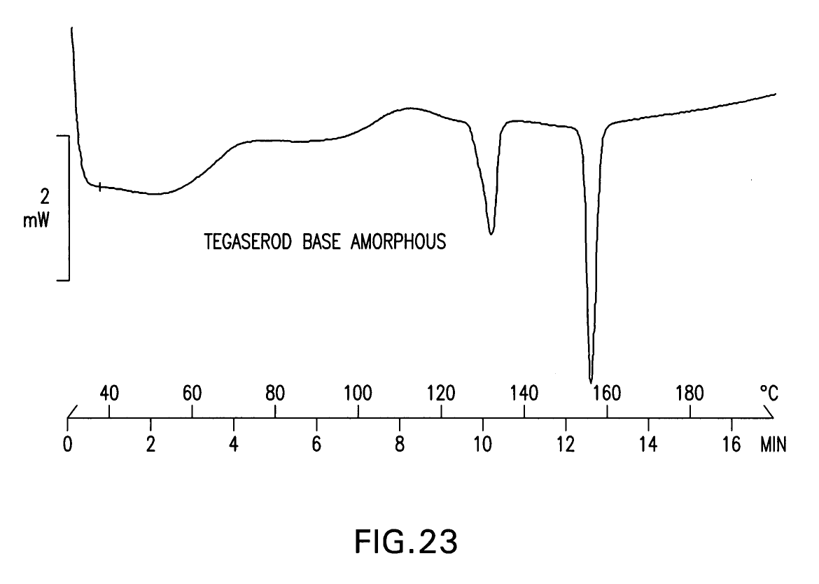

FIG. 24 is a DSC curve of tegaserod acetate Form J.

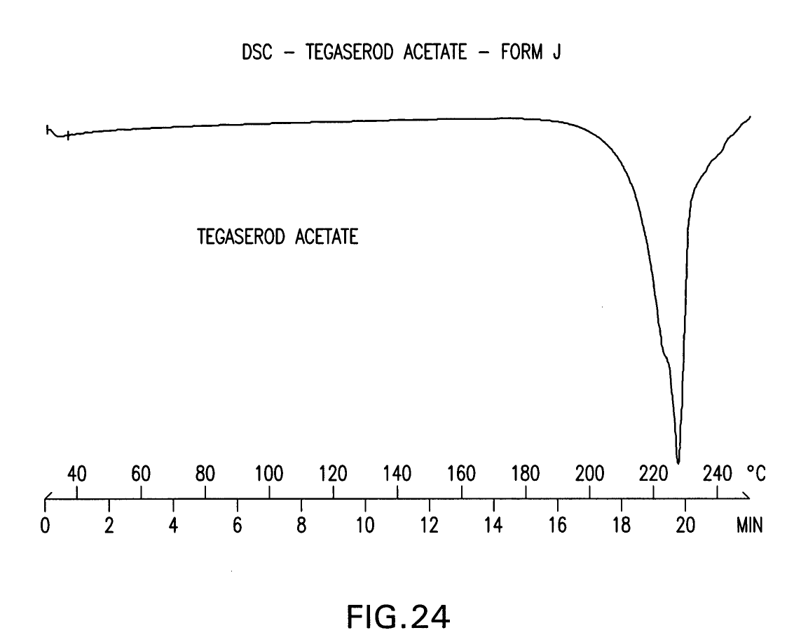

FIG. 25 is a DSC curve of tegaserod hemi-maleate.

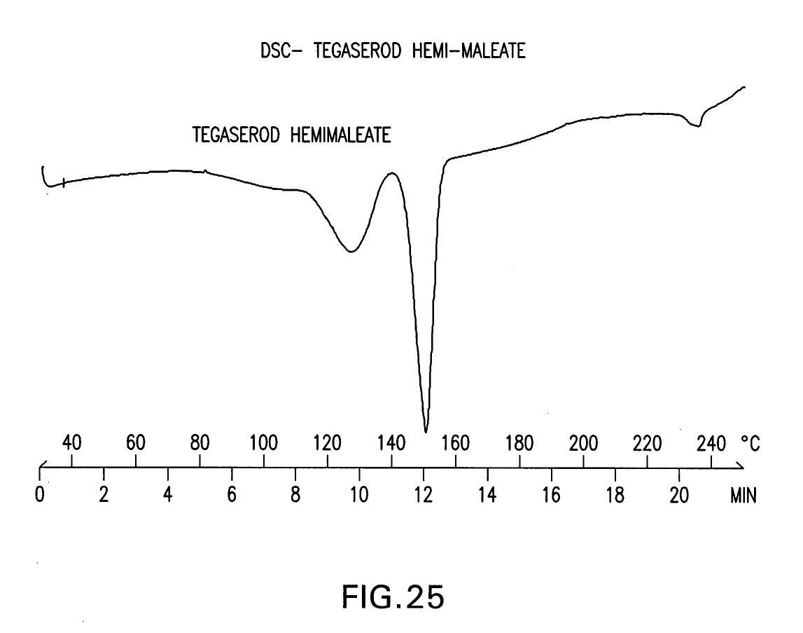

FIG. 26 is an X-Ray powder diffraction of tegaserod maleate Form A as published in IPCOM000021161D.

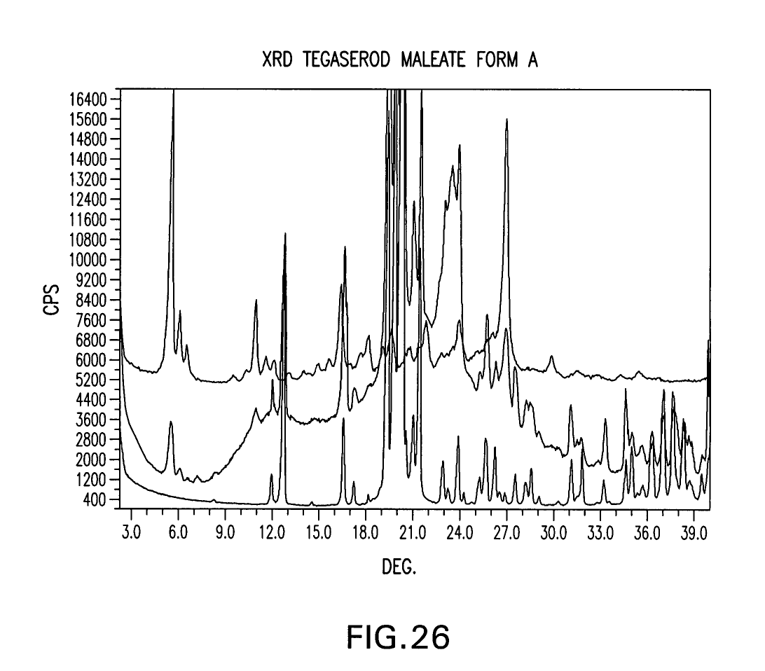

### DETAILED DESCRIPTION OF THE INVENTION

As used herein, the term “reduced pressure” refers to any pressure below one atmosphere. As used herein, the term “vacuum” refers to a pressure below about 50 mmHg, with about 30 mmHg or below being preferred. As used herein, the term slurry refers to a hetrogeneous mixture where complete dissolution does not occur.

The present invention provides tegaserod acetate. The tegaserod acetate provided by the present invention may be in the solid state, and may also be crystalline.

The present invention further provides for polymorphic forms of tegaserod maleate, acetate and base, and processes for their preparation. The typical X-Ray powder diffraction peaks of each form are shown in the following table. The most typical peak positions (degrees 2-theta) of each form are marked in bold.

| TEGASEROD MALEATE FORMS ±0.2 | | | | | | | | | | |
|---|---|---|---|---|---|---|---|---|---|---|
| degrees 2theta | | | | | | | | TEGASEROD | | TEGASEROD |
| | | | | | | | Hemi- | BASE FORMS | | ACETATE |
| B | B1 | B2 | B3 | C | D | E | maleate | F | H | J |
| 7.1 | 10.3 | 7.0 | 7.2 | 7.8 | 11.1 | 7.9 | 5.0 | 10.2 | 7.7 | 7.3 |
| 7.9 | 13.9 | 8.0 | 8.0 | 8.7 | 14.6 | 10.3 | 9.9 | 11.3 | 8.8 | 8.7 |
| 10.2 | 15.5 | 8.7 | 10.3 | 10.3 | 17.1 | 15.9 | 14.2 | 15.3 | 11.9 | 10.9 |
| 15.7 | 16.1 | 10.4 | 15.6 | 15.6 | 17.7 | 16.6 | 14.8 | 16.9 | 15.1 | 13.5 |
| 16.9 | 16.5 | 15.6 | 16.0 | 17.1 | 20.2 | 17.1 | 19.8 | 18.3 | 16.0 | 18.2 |
| 17.2 | 17.1 | 16.0 | 16.8 | 17.3 | 21.6 | 19.5 | 20.8 | 19.2 | 16.8 | 18.9 |
| 19.5 | 19.5 | 16.8 | 17.3 | 19.6 | 22.6 | 20.6 | 21.5 | 20.3 | 17.6 | 21.8 |
| 20.7 | 20.3 | 17.0 | 19.6 | 21.7 | 23.8 | 21.4 | 23.1 | 21.3 | 18.1 | 23.1 |
| 21.6 | 20.9 | 17.3 | 20.7 | 22.8 | 24.9 | 22.0 | 23.8 | 21.8 | 19.3 | 24.4 |
| 23.2 | 22.0 | 19.7 | 21.6 | 23.2 | 25.2 | 22.4 | 25.9 | 22.7 | 21.8 | |
| 24.1 | 23.1 | 20.6 | 22.5 | 23.7 | 26.0 | 23.4 | | 24.4 | 22.7 | |
| 24.6 | 24.2 | 21.3 | 23.3 | 25.1 | 27.3 | 24.4 | | 27.6 | 23.9 | |
| 25.2 | 25.3 | 22.2 | 24.5 | 27.1 | 28.6 | 25.4 | | 29.6 | 25.4 | |
| 25.9 | 26.7 | 23.2 | 25.5 | | 29.3 | 26.0 | | 31.1 | 26.5 | |
| 27.8 | 27.9 | 25.3 | 26.0 | | 31.0 | 28.0 | | 32.7 | 29.8 | |
| 28.8 | 28.7 | 27.1 | 27.2 | | 33.9 | 28.5 | | | | |
| 29.4 | 30.4 | 27.9 | 28.0 | | 35.8 | 29.3 | | | | |
| 30.7 | | 29.1 | 29.3 | | | | | | | |

These forms are essentially free from other forms, i.e., they contain no more than 5% of other forms. Polymorphic purity may be tested by XRD, with the area under the peaks used to calculate polymorphic purity.

Tegaserod Maleate Form A

A typical DSC of tegaserod maleate Form A has a characteristic endothermic peak at about 185-188° C. Form A may be prepared by crystallization out of a solution of tegaserod maleate in a suitable solvent. Examples of suitable solvents include dipolar aprotic solvents (such as DMSO, DMF, acetonitrile), C₁-C₄ alkyl acetates (such as ethyl lactate, butyl lactate), C₃-C₇ alkyl ketones (such as methylethylketone), C₁-C₄ alcohols (such as ethanol, n-propanol, isopropanol and butanol), dioxane, halo(C₁-C₄) alkanes (including chlorinated C₁-C₄ hydrocarbons such as chloroform and dichloromethane), ethoxyethanol, 2-ethoxyethanol, pyrrolidone and C₁-C₄ alkylsubstituted pyrrolidones (e.g. 1-methyl-2-pyrrolidone), water and N,N-dimethylacetamide and mixtures thereof. In one embodiment, Form A is recovered without addition of an anti-solvent. In this embodiment, the solution is preferably cooled to a temperature of about 10 EC to about 40° C., more preferably room temperature, to induce crystallization. Water may also be used as a co-solvent to prepare the solution followed by cooling to induce crystallization.

In another embodiment, water is added as an antisolvent to a solution of tegaserod maleate in an appropriate solvent to induce crystallization.

Form A may also be prepared by crystallization/precipitation by combining a solution of maleic acid in a solvent with a solution of tegaserod free base in the same or different solvent. Preferably the solvent is acetonitrile, n-butanol, dioxane, methyl ethyl ketone, ethyl lactate, ethyl acetate or water.

Tegaserod Maleate Forms B, B1, B2 and B3

Tegaserod Forms B, B1, B2 and B3 are related in that all of them have a characteristic endothermic peak at about, 140° C. which signifies a desolvation and transformation to Form A. Form B is a solvated form of 1-propanol (Syn. n-propanol) Form B1 is a solvated form of CHCl₃, and Forms B2 and B3 are solvated forms of ethanol. The term solvate refers to compounds having solvents incorporated into the crystalline structure.

Tegaserod Maleate Form B

The typical DSC curve of tegaserod form B shows one endothermic peak at about 140° C. due to desolvation of 1-propanol and transformation to Form A, and one endothermic peak at about 185 to about 188° C. due to melting of Form A. Form B is a 1-propanol solvated form (about 7% weight loss by TGA, which corresponds to hemi-1-propanolate-stochiometric value for hemipropanolate: 6.7%).

Tegaserod maleate Form B is generally prepared through a slurry of tegaserod maleate Form A in a suitable solvent, preferably n-propanol. The propanol may be in a mixture with water, preferably up to about 20% of water by volume.

Tegaserod Maleate Form B1.

The typical DSC curve of tegaserod Form B1 shows one endothermic peak at about 140° C. due to desolvation and transformation to Form A, and one endothermic peak at about 185-188° C. due to melting of Form A. Tegaserod maleate Form B1 is a CHCl₃ solvate (theoretical value: 8.8%). Form B1 shows a TGA curve with 10.8% weight loss step. Form B1 contains 9.2% CHCl₃ and 1% EtOH as residual solvents, as measured by GC. The integration of ¹H NMR of Form B1 showed a ratio of 0.3:1 (CHCl₃/Tegaserod).

Tegaserod maleate Form B1 may be prepared by crystallization out of chloroform, optionally in a mixture with a C₁ to C₄ alcohol. In a preferred embodiment, crystallization is induced by lowering of the temperature to about 30 to 50 EC. A mixture that may be used is that of chloroform and ethanol.

Tegaserod Maleate Form B2

The typical DSC curve of tegaserod form B2 shows one endothermic peak at about 140° C. due to desolvation and transformation to Form A, and one endothermic peak at about 185-188° C. due to melting of Form A. Form B2 contains 0.9% water (by karl fisher) and shows a TGA curve with 4% weight loss step. Form B2 is an ethanolate (theoretical value of 1/3 ethanolate is 3.5%).

Tegaserod maleate Form B2 may be prepared by slurry of another form of tegaserod maleate, preferably Form A, in ethanol, optionally in a mixture with water. A preferred mixture is up to about 20% water by volume, with about 5% water being preferred. The slurry process may be carried out at room temperature for about 12 to 24 hours.

Form B2 may also be prepared from a slurry or solution of tegaserod maleate an ethanol containing solution. The slurry or solution may be cooled in order to induce crystallization. The ethanol solution preferably contains at least about 80% ethanol by volume, and preferably at least one of methanol, ethyl acetate and water.

Tegaserod Maleate Form B3

The typical DSC curve of tegaserod Form B3 shows one endothermic peak at about 140° C. due to desolvation and transformation to Form A, and one endothermic peak at about 185-188° C. due to melting of Form A. Form B3 is also an ethanol solvated form (about 5% weight loss by TGA, which corresponds to hemi-ethanolate—stochiometric value for hemi-ethanolate: 5.2%).

Form B3 may generally be prepared by crystallization from an ethanol containing solution, slurry in ethanol or absorption of ethanol vapors. The solution may contain preferably at least about 80% ethanol, and preferably at least one of methanol, ethyl acetate and water.

Tegaserod Maleate Form C

The typical DSC curve of tegaserod Form C shows one or more small endothermic peaks below 160° C. and a multiple event above 170° C. due to a transformation to Form A, and one endothermic peak at about 185-188° C. due to melting of Form A. Form C may contain up to 2% water (by Karl Fisher).

Form C may also be prepared by drying Form B2 under ambient or reduced pressure, preferably under reduced pressure at a temperature of about 30° C. to about 50° C. for about 12 to 24 hours.

Tegaserod Maleate Form D

Tegaserod maleate Form D may be prepared by slurry or crystallization from 1-methyl-2-pyrrolidone, n-propanol or mixtures thereof. The slurry is preferably continued for a day at about room temperature. Crystallization of Form D is preferably carried out without rapid precipitation by addition of an anti-solvent, in that such process may result in formation of Form A.

Heating of Form D causes a transformation to Form A. Form D is preferably heated to a temperature of at least about 30 EC, more preferably about 40 EC for about a day. Since Form A exhibits thermal stability at higher temperatures, it is possible to use higher temperatures to cause the transformation. The term “stable” as used herein refers to a polymorphic change of less than about 5% by weight, more preferably less than about 2% by weight.

Tegaserod Maleate Form E

The typical DSC curve of tegaserod Form E shows one endothermic peak at about 130° C. due to a solid-solid transformation to Form A and one endothermic peak at about 185-188° C. due to melting of Form A. Form E may be a dioxane solvated form (about 9.5% weight loss by TGA, which corresponds to hemi-dioxane solvate—stochiometric value: 9.5%).

Tegaserod maleate Form E may generally be prepared by slurry of tegaserod Form A in dioxane. The slurry process is preferably carried out at a temperature of about 20° C. to about 30° C. for about 12 to 24 hours. Tegaserod maleate Form E may also be prepared by combining tegaserod base with a solution of maleic acid in THF.

Tegaserod Hemi-maleate

The other forms of tegaserod maleate described herein have a 1:1 molar ratio of tegaserod to maleate. We have also discovered an additional form that is a hemi-maleate, i.e., it has a 2:1 molar ratio of tegaserod to maleate.

The typical DSC curve of tegaserod hemi-maleate shows a broad endotherm below 140 EC due to solvent desorption, and a melting endotherm at about 150 degrees.

Tegaserod hemi-maleate is a hemihydrate (about 2.5% weight loss of water by both TGA and Karl Fisher, which corresponds to the hemihydrate).

In addition, the structure was confirmed according to an HPLC assay: tegaserod in the sample was measured to be 81.18% (calculated 81.79%) and maleic acid in the sample was measured to be 16.01% (calculated value: 15.76%).

Tegaserod hemi-maleate may be prepared by combining tegaserod base with a solution of maleic acid in ethyl acetate and water. The reaction mixture is preferably heated, more preferably to at least about 40° C., and most preferably to at least about 65° C. Preferably, ethyl acetate:water ratio is about 97:1 to about 97:5, more preferably about 97:3 (v/v). The hemi-maleate is recovered as a precipitate.

Tegaserod hemi-maleate prepared by this process may be recovered by filtration, and dried at a temperature of at least 40° C. in a vacuum oven for about 12 to about 24 hours.

Tegaserod Base Form F

The typical DSC curve of tegaserod base Form F has one endothermic peak at about 154° C.

Tegaserod base Form F may generally be prepared by dissolving tegaserod in a chlorinated hydrocarbon (C₁ to C₈ are the practical hydrocarbons), e.g. chloroform and dichloromethane, preferably dichloromethane; and removing the chlorinated hydrocarbon. Removing is preferably carried out by evaporation. The process may further comprise preliminary steps of distributing tegaserod maleate between an aqueous phase and the hydrocarbon, contacting the maleate with a base, and recovering the hydrocarbon containing tegaserod. Weak bases such as amines are preferred. Most preferred bases are C1 to C6 dialkylamines. Preferably the chlorinated hydrocarbon is dichloromethane. Optionally the removing step is carried out under reduced pressure.

Tegaserod Base Form H

The typical DSC curve of tegaserod base Form H has two endothermic peaks. The first appears at about 134° C., and the second at about 156° C., probably due to polymorphic conversion. Tegaserod maleate Form H may generally be prepared by precipitation, such as by dissolving tegaserod base in a C₁ to C₄ alcohol, combining the alcohol with an antisolvent and recovering the crystalline form as a precipitate. Preferably the alcohol is ethanol and the anti-solvent is water.

Tegaserod base Form H may also be prepared by slurry in ethyl acetate under conditions such that ethyl acetate does not hydrolyze.

Tegaserod Base Amorphous

The typical DSC curve of tegaserod base amorphous has broad endotherms below about 100° C. and two endothermic peaks at about 132° C. and 156° C. Amorphous tegaserod base may be prepared by solvent removal from a solution of tegaserod in a C₁ to C₄ alcohol, preferably methanol or ethanol. Preferably, solvent removal is carried out by evaporation. The evaporation process may be accelerated by heating and reUucing the pressure. Evaporation is preferably carried out under vacuum at a temperature of about 50 EC to about 70 EC until no solvent is observed.

Amorphous tegaserod maleate of the present invention preferably contains less than about 20% crystalline tegaserod, more preferably less than 10%, wt/wt, and most preferably less than about 5% wt/wt. Presence of amorphous form may be detected by lack of peaks in a powder XRD pattern or lack of a melting point in a DSC thermogram. The area under the peaks in an XRD pattern may be added to obtain total amount of crystalline material. With DSC, presence of endotherms may point to melting of crystalline material.

Tegaserod Acetate Form J

The present invention also provides for tegaserod acetate. Tegaserod acetate has not previously been reported in the literature. The typical DSC curve of tegaserod Form J does not show any melting point in the range of 140 degrees. Form J of tegaserod acetate is anhydrous (less than about 0.1% weight loss by TGA).

Additionally, tegaserod acetate Form J was characterized by elemental analysis; Anal. Calcd for C₁₈H₂₇N₅O₃: C, 59.81; H, 7.53; N, 19.38. Found: C, 59.64; H, 7.49; N, 19.34. Tegaserod acetate Form J is less soluble than tegaserod base.

Tegaserod acetate Form J may be prepared by mixing tegaserod base or tegaserod maleate or another salt of tegaserod in the presence of a base, with ethyl acetate under conditions where the ethyl acetate hydrolyzes, to form a slurry, and recovering the crystalline form. Hydrolysis for example can be induced by the tegaserod base present in the reaction mixture. Alternatively, acetic acid may be used instead of ethyl acetate. Form J may also be obtained by heating the mixture of tegaserod base and ethyl acetate to a high temperature such as reflux.

Heating

It was found that forms B, B2 and C transform to Form A upon heating:

| Temperature/ | | | | |
|---|---|---|---|---|
| Polymorph form | 2 hr. | 2 hr. | 2 hr. | 2 hr. |
| before heating | 80 deg. | 100 deg. | 120 deg. | 140 deg. |
| A | A | A | A | A |
| B2 | B + C + A | B + C + A | C + A | C + A |
| C | C | C | C | C > A |
| B (wet) | B | B > C + A | A > C | A |
| B (wet) | B | B > A | | |

Form B nmay transform to Form C upon heating before it transforms to Form A

It is assumed that Forms B1, B3, E transform to Form A upon heating, from observation of the DSC curves, in which all the forms have the melting peak of Form A.

Another form that shows a transformation with heating is Form D. Form D transforms to Form A upon heating.

The starting material used for the processes of the present invention may be any crystalline or amorphous form of tegaserod base or maleate, including various solvates and hydrates. With crystallization processes, the crystalline form of the starting material does not usually affect the final result. With trituration, the final product may vary depending on the starting material. One of skill in the art would appreciate the manipulation of the starting material within skill in the art to obtain a desirable form with trituration. The present invention is not limited to the starting form used for trituration unless if such form is essential for obtaining another form.

Many processes of the present invention involve crystallization out of a particular solvent, i.e., obtaining a solid material from a solution. One skilled in the art would appreciate that the conditions concerning crystallization may be modified without affecting the form of the polymorph obtained. For example, when mixing tegaserod or its maleate in a solvent to form a solution, warming of the mixture may be necessary to completely dissolve the starting material. If warming does not clarify the mixture, the mixture may be diluted or filtered. To filter, the hot mixture may be passed through paper, glass fiber or other membrane material, or a clarifying agent such as celite. Depending upon the equipment used and the concentration and temperature of the solution, the filtration apparatus may need to be preheated to avoid premature crystallization.

The conditions may also be changed to induce precipitation. A preferred way of inducing precipitation is to reduce the solubility of the solvent. The solubility of the solvent may be reduced, for example, by cooling the solvent.

In one embodiment, an anti-solvent is added to a solution to decrease its solubility for a particular compound, thus resulting in precipitation. Another way of accelerating crystallization is by seeding with a crystal of the product or scratching the inner surface of the crystallization vessel with a glass rod. Other times, crystallization may occur spontaneously without any inducement. The present invention encompasses both embodiments where crystallization of a particular form of tegaserod occurs spontaneously or is induced/accelerated, unless if such inducement is critical for obtaining a particular form.

A solid may be recovered from a reaction mixture in a routine fashion such as by filtration, centrifugation or decanting.

Tegaserod maleate or base of defined particle size may be produced by known methods of particle size reduction starting with crystals, powder aggregates and course powder of the new crystalline forms of tegaserod maleate. The principal operations of conventional size reduction are milling of a feedstock material and sorting of the milled material by size.

A fluid energy mill, or micronizer, is an especially preferred type of mill for its ability to produce particles of small size in a narrow size distribution. As those skilled in the art are aware, micronizers use the kinetic energy of collision between particles suspended in a rapidly moving fluid stream to cleave the particles. An air jet mill is a preferred fluid energy mill. The suspended particles are injected under pressure into a recirculating particle stream. Smaller particles are carried aloft inside the mill and swept into a vent connected to a particle size classifier such as a cyclone. The feedstock should first be milled to about 150 to 850 μm which may be done using a conventional ball, roller, or hammer mill. The polymorphic forms forms of the present invention have a maximal particle size of below about 250μ, more preferably below about 200μ, most preferably below about 100μ. One of skill in the art would appreciate that some crystalline forms may undergo a transition to another form during particle size reduction.

Pharmaceutical compositions may be prepared as medicaments to be administered orally, parenterally, rectally, transdermally, bucally, or nasally. Suitable forms for oral administration include tablets, compressed or coated pills, dragees, sachets, hard or gelatin capsules, sub-lingual tablets, syrups and suspensions. Suitable forms of parenteral administration include an aqueous or non-aqueous solution or emulsion, while for rectal administration suitable forms for administration include suppositories with hydrophilic or hydrophobic vehicle. For topical administration the invention provides suitable transdermal delivery systems known in the art, and for nasal delivery there are provided suitable aerosol delivery systems known in the art.

Pharmaceutical formulations of the present invention contain the above disclosed polymorphic forms of tegaserod base or maleate. The pharmaceutical composition may contain only a single form of tegaserod base, maleate or acetate, or a mixture of variotus forms of tegaserod maleate, with or without amorphous form. In addition to the active ingredient(s), the pharmaceutical compositions of the present invention may contain one or more excipients or adjuvants. Selection of excipients and the amounts to use may be readily determined by the formulation scientist based upon experience and consideration of standard procedures and reference works in the field.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose fimctions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants ean be added to improve the flowability of a non-compacted solid composition and to improve the accuracy of dosing. Excipients that may function as glidants include colloidal silicon dixoide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate. Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, the active ingredient and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the present invention, a liquid composition may also contain a buffer such as gluconic acid, lactic acid, citric acid or acetic acid, sodium gluconate, sodium lactate, sodium citrate or sodium acetate.

Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and losenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules.

Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

The dosage used is preferably from about 1 mg to about 10 mg of tegaserod base equivalent, more preferably from about 2 to about 6 mg. The pharmaceutical compositions of the present invention, used to treat irritable bowel syndrome in a mammal such as a human, are preferably in the form of a coated tablet, and are administered on an empty stomach twice a day, for a period of about 4 to about 6 weeks. Additional administration may occur if the patient responds positively to the treatment. Generally, each 1.385 mg of tegaserod as the maleate is equivalent to 1 mg of tegaserod free base. A possible formulation is as follows: crospovidone, glyceryl monostearate, hydroxypropyl methylcellulose, lactose monohydrate, poloxamer 188, and polyethylene glycol 4000.

### Instruments

X-Ray powder diffraction data were obtained using by method known in the art using a SCINTAG powder X-Ray diffractometer model X'TRA equipped with a solid state detector. Copper radiation of 1.5418 Å was used. A round aluminum sample holder with zero background was used. All peak positions are within ±0.2 degrees two theta.

DSC analysis was done using a Mettler 821 Star^{e}. The weight of the samples is about 3-6 mg; the samples were scanned at a rate of 10° C./min from 30° C. to at least 200° C. The oven is constantly purged with nitrogen gas at a flow rate of 40 ml/min. Standard 40 μl aluminum crucibles covered by lids with 3 holes were used. TGA analysis was done using a Mettler M3 thermogravimeter. The weight of the samples is about 10 mg; the samples were scanned at a rate of 10° C./min from 25° C. to 200° C. The oven is constantly purged with nitrogen gas at a flow rate of 40 ml/min. Standard 150 μl alumina crucibles covered by lids with 1 hole were used.

Karl Fisher analysis was performed according to the known art.

### EXAMPLES

Tegaserod Maleate Form A

### Example 1

### General Method for the Preparation of Tegaserod Maleate Form A from Crystallization

Tegaserod maleate (1 g) was combined with the appropriate solvent (5 mL), and heated to reflux. Then, additional solvent was added until complete dissolution. After the compound was dissolved, the oil bath was removed and the solution was cooled to room temperature. The solid was filtrated and washed with 5 mL of the same solvent and dried in a vacuum oven at 40° C. for 16 hours.

| | Total | Form | Form |
|---|---|---|---|
| | Volume | before | After |
| Solvent | (mL) | Drying | Drying |
| Acetonitrile | 80 | A | A |
| Butyl lactate | 10 | A | A |
| Methyl ethyl ketone | 60 | A | A |
| sec-butanol | 40 | A | A |
| Dioxane | 120 | A | A |
| Methanol/water 20:80 | 60 | A | A |
| Ethanol/water 20:80 | 60 | A | A |
| Isopropanol/water 1:1 | 7 | A | A |
| Isopropanol/water 20:80 | 43 | A | A |
| Acetonitrile/water 1:1 | 7 | A | A |
| Acetonitrile/water 20:80 | 47 | A | A |
| Chloroform/2- | 7 | A | A |
| ethoxyethanol 1:1 | | | |
| Chloroform/2- | 13 | A | A |
| ethoxyethanol 25:75 | | | |
| Water/2-ethoxyethanol 1:1 | 5 | A | A |
| n-BuOH | 6 | A | A |
| Water/1-methyl-2- | 8 | D | A |
| pyrrolidone 75:25 | | | |

### Example 2

### General Method for the Preparation of Tegaserod Maleate Form A from Precipitation

To a solution of tegaserod maleate (1 g) in the appropriate solvent was added 3 mL of water. The resulting solid was filtrated and washed with water and dried in a vacuum oven at 40° C. for 16 hours. The product was analyzed by XRD and found to be Form A before and Form A after the drying.

| Solvent | Volume (mL) |
|---|---|
| Dimethyl sulfoxide | 5 |
| N,N-Dimethylformamide | 2.5 |
| 1-methyl-2-pyrrolidone | 2.5 |
| N,N-Dimethylacetamide | 3 |

### Example 3

### Preparation of Tegaserod Maleate Form A by Drying

Tegaserod maleate Form D was heated at 40° C. in a vacuum oven for about 16 hours.

### Example 4

### Preparation of Tegaserod Maleate Form A

A solution of maleic acid (0.85 g) in 10 mL methanol was added to a solution of Tegaserod free base (2 g) in methanol (40 mL) at room temperature followed by 30 minutes stirring. The solid was then filtrated and washed with methanol and dried and recrystallized in acetonitrile. Drying in vacuum oven at 40° C. for 16 hours gives 1.95 g (70% yield). Tegaserod maleate Form A was characterized by ¹H and 13C-NMR according to the literature.

### Example 5

### Preparation of Tegaserod Maleate Form A in Ethyl Acetate/Water

To a solution of 6.03 g of tegaserod free base in 50 mL ethyl acetate, was added a solution of maleic acid (2.74 g in 25 mL water). The resulting solid was filtered off, washed with water, dried in a vacuum oven at 40° C. for 16 hours, and was found to be Form A.

Tegaserod Maleate Form B

### Example 6

### General Method for the Preparation of Tegaserod Maleate Form B by Slurry

A slurry of tegaserod maleate Form A (1 g) in 7 mL of the appropriate solvent was stirred at 20-30° C. for 24 hours. The solid was filtrated and washed with 1 mL of same solvent. The material was dried for 16 hours as indicated in the table and analyzed. The product was analyzed by XRD and found to be Form B before and Form B after the drying.

| Solvent | Drying conditions |
|---|---|
| n-propanol/water 95:5 | 40° C. under vacuum |
| n-propanol | 40° C. under vacuum |
| n-propanol | 50° C. under atmospheric press. |

Tegaserod Maleate Form B1

### Example 7

### General Method for the Preparation of Tegaserod Maleate Form B1 by Crystallization

A slurry of tegaserod maleate (1 g) in the appropriate solvent (5 mL) was heated to reflux, and then additional solvent was added until complete dissolution. After the compound was dissolved, the oil bath was removed and the solution was cooled to room temperature. The solid was filtrated and washed with 5 mL of the same solvent and dried in a vacuum oven at 40° C. for 16 hours (except where is indicated). The product was analyzed by XRD and found to be Form B1 before and Form B1 after the drying.

| | Solvent | Total Volume (mL) |
|---|---|---|
| 1 | Chloroform/methanol 70:30 | 33 |
| 2 | Chloroform/ethanol 70:30 | 33 |

Tegaserod Maleate Form B2

### Example 8

### General Method for the Preparation of Tegaserod Maleate Form B2 by Slurry

A slurry of tegaserod maleate Form A (1 g) in the appropriate solvent was stirred at 20-30° C. for 24 hours. The solid was filtrated and washed with 1 mL of same solvent and the wet material was analyzed by XRD.

| Exp. | Solvent | Volume (mL) |
|---|---|---|
| 1 | ethanol¹ | 7 |
| 2 | ethanol¹/water 95:5 | 7 |

¹Denatured ethanol (contains 5% water and 5% methanol)

### Example 9

### General Method for the Preparation of Tegaserod Maleate Form B2 by Crystallization

A slurry of tegaserod maleate (1 g) in the appropriate solvent (5 mL) was heated to reflux, and then, additional solvent was added until complete dissolution. After the compound was dissolved, the oil bath was removed and the solution was cooled to room temperature. The solid was filtrated and washed with 5 mL of the same solvent and dried in a vacuum oven at 40° C. for 16 hours.

| | Total | Form | Form |
|---|---|---|---|
| | Volume | before | After |
| Solvent | (mL) | drying | Drying |
| Ethanol¹ | 22 | B2 | B2 |
| Ethanol¹/water 1:1 | 15 | B2 | B2 |
| Ethanol¹/water 80:20 | 7 | B2 | B2 |
| Ethanol¹/ethyl acetate 60:40 | 18 | B2 | B2 + C |

¹Denatured ethanol (contains 5% water and 5% methanol)

Tegaserod Maleate Form B3

### Example 10

### Preparation of Tegaserod Maleate Form B3 from Tegaserod Maleate Form A by Slurry

A slurry of tegaserod maleate Form A (1 g) in 7 mL of ethanol was stirred at 20-30° C. for 24 hours. The solid was filtrated and washed with 1 mL of ethanol. The wet material was analyzed by XRD and found to be Form B3.

### Example 11

### General Method for the Preparation of Tegaserod Maleate Form B3 by Crystallization

A slurry of tegaserod maleate Form A (1 g) in the appropriate solvent (5 mL) was heated to reflux, and then additional solvent was added until complete dissolution. After the compound was dissolved, the oil bath was removed (except where is indicated) and the solution was cooled to room temperature. The solid was filtrated and washed with 5 mL of the same solvent. The wet material was analyzed by XRD and found to be Form B3.

| Solvent | Total Volume (mL) |
|---|---|
| Ethanol¹ | 30 |
| methanol/ethanol² 1:1 | 20 |
| water/ethanol² 20:80 | 7 |
| ethanol³ | 22 |

¹Dried at 50° C. under atmospheric pressure.
²Denatured ethanol (contains 5% water and 5% methanol).
³The solution was cooled down in the presence of the oil bath.

Tegaserod Maleate Form C

### Example 12

### Preparation of Tegaserod Maleate Form C by Drying

Tegaserod maleate Form B2 was heated at 40° C. in a vacuum oven for about 16 hours to produce tegaserod maleate Form C.

Tegaserod Maleate Form D

### Example 13

### Preparation of Tegaserod Maleate Form D by Slurry

A slurry of tegaserod maleate Form A (1 g) in 7 mL n-propanol was stirred at 20-30° C. for 24 hours. The solid was filtrated and washed with 1 mL of same solvent and the wet material was analyzed by XRD and found to be form D.

### Example 14

### General Method for the Preparation of Tegaserod Maleate Form D by Crystallization

A slurry of tegaserod maleate (1 g) in the appropriate solvent (5 mL) was heated to reflux, and then, additional solvent was added until complete dissolution. After the compound was dissolved, the oil bath was removed and the solution was cooled to room temperature. The solid was filtrated and washed with 5 mL of the same solvent and the wet material was analyzed.

| Solvent | Total Volume (mL) |
|---|---|
| n-propanol | 25 |
| 1-methyl-2-pyrrolidone | 8 |

Tegaserod Maleate Form E

### Example 15

### Preparation of Tegaserod Maleate Form E by Slurry

A slurry of tegaserod maleate Form A (1 g) in 7 mL dioxane was stirred at 20-30° C. for 24 hours. The solid was filtrated and washed with 1 mL of same solvent and dried in a vacuum oven at 40° C. for 16 hours.

Tegaserod Free Base Form F

### Example 16

### Preparation of Tegaserod Free Base Form F

Tegaserod maleate (50 g) was added to a mixture of CH₂Cl₂ (750 mL) and water (750 mL) followed by 61 mL of diethyl amine. The mixture was stirred for an additional half hour and the insoluble solids removed by filtration. The organic phase was separated and washed with water and the solvent evaporated. The resulting white solid was washed with 100 mL of CH₂Cl₂ and the solvent evaporated. Drying in vacuum oven at 40° C. for 16 hours gives 23 g (64% yield). Tegaserod base was characterized by ¹H and ¹³C-NMR.

Tegaserod Free Base Form H

### Example 17

### Preparation of Tegaserod Free Base Form H by Precipitation

To a solution of tegaserod free base (1 g) in absolute ethanol (30 mL) was added 50 mL of water. The resulting solid was stirred for half an hour, filtrated and washed with water (2 mL) and dried in a vacuum oven at 40° C. for 16 hours.

### Example 18

### Preparation of Tegaserod Base Form H by Slurry

A slurry of tegaserod free base Form F (6 g) in 50 mL ethyl acetate was stirred at 5-10° C. for 24 hours. The solid was filtrated and washed with 15 mL of same solvent and dried in a vacuum oven at 40° C. for 16 hours.

Tegaserod Acetate Form J

### Example 19

### Preparation of Tegaserod Acetate Form J at Room Temperature

A slurry of tegaserod base amorphous (6 g) in 50 mL ethyl acetate was stirred at 20-30° C. for 24 hours. The solid was filtrated and washed with 15 mL of same solvent and dried in a vacuum oven at 40° C. for 16 hours.

### Example 20

### Preparation of Tegaserod Acetate Form J at Reflux Temperature

A slurry of tegaserod base amorphous (6 g) in 50 mL ethyl acetate was stirred at reflux for 24 hours. The solid was filtrated and washed with 15 mL of same solvent and dried in a vacuum oven at 40° C. for 16 hours.

### Example 21

### Preparation of Tegaserod Acetate Form J

To a slurry of tegaserod maleate Form A (15 g) in EtOAc (210 mL) and water (210 mL) was added 38.4 g of NaOH 47%. The mixture was stirred overnight and the resulting white solid was isolated by filtration and washed with 100 mL of water. Drying in vacuum oven at 40° C. for 16 hours gives 12.38 g (90% yield). Tegaserod acetate was characterized by ¹H and ¹³C-NMR.

### SYNTHETIC PROCESSES

Tegaserod free base was prepared according to the patent EP505322 B1.

### Example 22

### Preparation of Crystalline Tegaserod Maleate

A solution of maleic acid (0.85 g ) in 10 mL of the appropriate solvent was added to a solution of Tegaserod free base (2 g) dissolved in the appropriate solvent (at the indicated volume) at room temperature followed by 30 minutes stirring. The solid was then filtrated and washed with methanol and dried in vacuum oven at 40° C. for 16 hours. Tegaserod maleate was characterized by ¹H and ¹³C-NMR according to the literature. The reactions performed in different solvents proceed with the following chemical yields:

| Solvent (Volume mL) | Chemical yield | Polymorphic Forms |
|---|---|---|
| ethanol (60) | 98% | B3 |
| iso-propanol (200) | 93% | A + E |
| n-propanol (100) | 93% | B1 + A |
| acetonitrile (300) | 85% | A |
| n-butanol (90) | 83% | A |
| Dioxane (100) | 46% | A > E |
| methyl ethyl ketone (70) | 56% | A |
| Tetrahydrofuran (40) | 83% | E >> A |
| ethyl lactate (25) | 46% | A |
| ethyl acetate (25) | 39% | A |

### Example 23

### Preparation of Tegaserod Hemi-maleate Hemihydrate

A solution of maleic acid (2.32 g in 22 mL ethyl acetate/water 97:3) was added to a mixture of tegaserod base in ethyl acetate, and the reaction mixture was heated to 65° C. and stirrer overnight. The resulting solid was filtered off and washed with water and ethyl acetate. Drying in vacuum oven at 40° C. for 16 hours gives 12.19 g of Tegaserod hemi-maleate hemihydrate. Depending on the base polymorph used a solution or slurry is obtained. When using amorphous tegaserod base, a solution is obtained, while when using any other base polymorph of tegaserod, a slurry is obtained.

Tegaserod Free Base Amorphous

### Example 24

### Preparation of Tegaserod Free Base Amorphous

Tegaserod free base (1 g) was dissolved in 40 mL methanol and evaporated to dryness at 60° C. under vacuum. The resulting solid was analyzed to yield amorphous tegaserod free base.

Having thus described the invention with reference to particular preferred embodiments and illustrative examples, those in the art may appreciate modifications to the invention as described and illustrated that do not depart from the spirit and scope of the invention as disclosed in the specification. The Examples are set forth to aid in understanding the invention but are not intended to, and should not be construed to, limit its scope in any way. The examples do not include detailed descriptions of conventional methods. Such methods are well known to those of ordinary skill in the art and are described in numerous publications. Polymorphism in Pharmaceutical Solids, Drugs and the Pharmaceutical Sciences, Volume 95 may be used as a guidance.

1. A process for preparing crystalline form of tegaserod maleate characterized by an X-ray Diffraction pattern having peaks at 5.4, 6.0, 6.6 and 10.8±0.2 degrees two theta (Form A) comprising:
c) preparing a solution of tegaserod maleate in an solvent; and
d) recovering the crystalline form as a precipitate.
wherein the solvent is selected from the group consisting of acetonitrile, butyl lactate, methyl ethyl ketone, butanol, dioxane, ethanol, isopropanol, chloroform, ethoxyethanol, 2-ethoxyethanol, pyrolidone, dimethyl sulfoxide, N,N-Dimethylformamide, 1-methyl-2-pyrrolidone, N,N-Dimethylacetamide, water and mixtures thereof, with the proviso that water is not used as an individual solvent.

2. The process of claim 1, wherein precipitation is induced by cooling the solution.

3. The process of claim 1, wherein precipitation is induced by adding an anti-solvent.

4. The process of claim 3, wherein the anti-solvent is water.

5. A process for preparing crystalline tegaserod maleate characterized by an X-ray Diffraction pattern having peaks at 5.4, 6.0, 6.6 and 10.8±0.2 degrees two theta (Form A) comprising heating a solvate of tegaserod maleate to cause desolvation.

6. A process for preparing crystalline form of tegaserod maleate characterized by an X-ray Diffraction pattern having peaks at 5.4, 6.0, 6.6 and 10.8±0.2 degrees two theta (Form A) comprising:
c) combining a solution of maleic acid in a solvent with a solution of tegaserod free base in the same or different solvent; and
d) recovering the crystalline form as a precipitate.
wherein the solvent is selected from the group consisting of acetonitrile, n-butanol, dioxane, methyl ethyl ketone, ethyl lactate, ethyl acetate and water.

7. A crystalline form of tegaserod maleate (Form B) characterized by an X-ray Diffraction pattern having peaks at 15.7, 16.9, 17.2, 24.1, 24.6 and 25.2±0.2 two theta.

8. The crystalline form of claim 7, further characterized by an X-ray Diffraction pattern having peaks at 7.1, 7.9, 19.5, 20.7, 21.6, 23.2, 24.1, 24.6, 25.2, 25.9, 27.8, 28.8, 29.4 and 30.7±0.2 two theta.

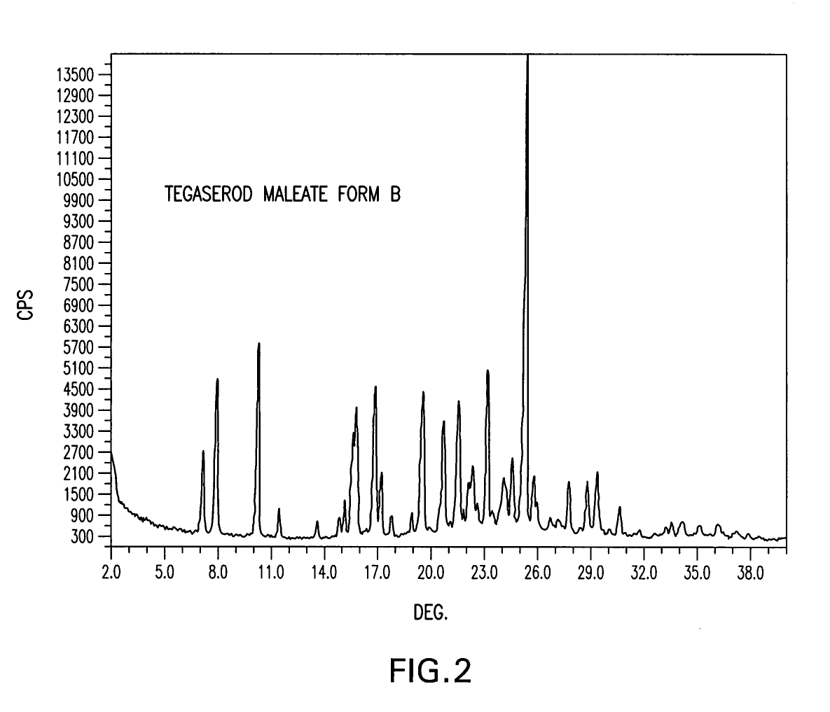

9. The crystalline form of claim 8, wherein the crystalline form has an X ray diffraction pattern as substantially depicted in FIG. 2.

10. The crystalline form of claim 7, wherein the crystalline form is 1-propanol solvate.

11. The crystalline form of claim 7, wherein the crystalline form has a particle size of below about 250μ and a polymorphic purity of at least about 95% as measured by area percentage XRD.

12. The crystalline form of claim 7, characterized by a DSC with an endothermic peak at about 140 EC, and another endothermic peak at about 185 to about 188 EC.

13. A process for preparing the crystalline form of claim 7 comprising slurrying a tegaserod maleate in solid state in 1-propanol, and recovering the crystalline form.

14. The process of claim 13, wherein the tegaserod maleate in the solid state is characterized by an X-ray Diffraction pattern having peaks at 5.4, 6.0, 6.6 and 10.8±0.2 degrees two theta (Form A).

15. A crystalline form of tegaserod maleate (Form B1) characterized by an X-ray Diffraction pattern having peaks at 10.3, 16.1, 16.5, 17.1, 20.3, 22.0, and 25.3±0.2 two theta.

16. The crystalline form of claim 15, further characterized by peaks at 13.9, 15.5, 19.5, 20.9, 23.1, 24.2, 26.7, 27.9, 28.7 and 30.4±0.2 two theta.

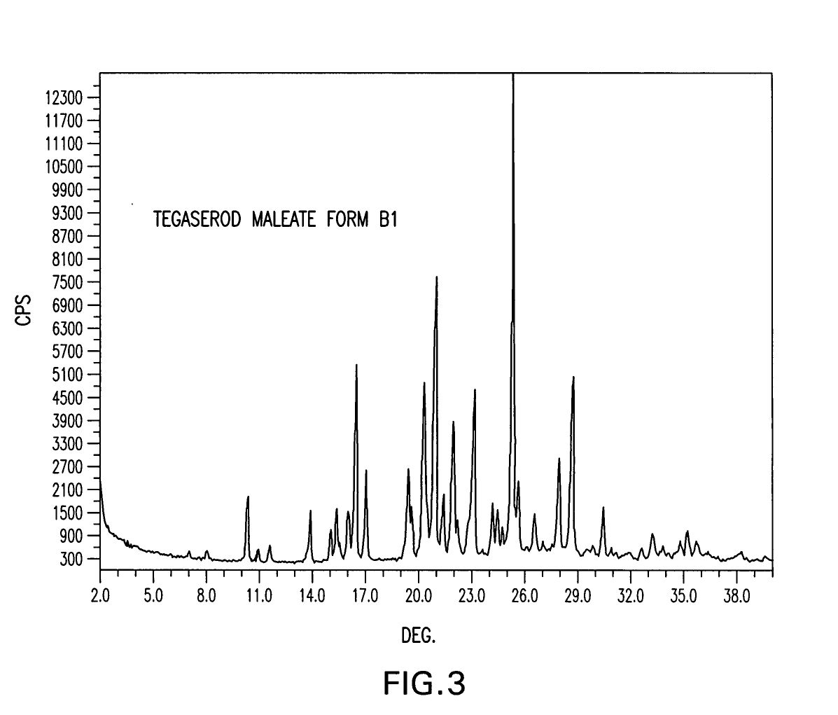

17. The crystalline form of claim 16, wherein the crystalline form has an X ray diffraction pattern as substantially depicted in FIG. 3.

18. The crystalline form of claim 15, wherein the crystalline form is a CHCl₃ solvate.

19. The crystalline form of claim 15, characterized by a DSC with an endothermic peak at about 140 EC, and another endothermic peak at about 185 to about 188 EC.

20. The crystalline form of claim 15, wherein the crystalline form has a particle size of below about 250μ and a polymorphic purity of at least about 95% as measured by area percentage XRD.

21. A process for preparing crystalline form of claim 15 comprising:
c) preparing a solution of tegaserod maleate in chloroform, optionally in mixture with methanol or ethanol; and
d) recovering the crystalline form as a precipitate.

22. Crystalline tegaserod maleate characterized by an X-ray Diffraction pattern having peaks at 8.7, 15.6, 16.0, 22.2, 25.3 and ±0.2 two theta (Form B2), wherein the crystalline form is an ethanolate solvate.

23. A process for preparing the crystalline form of claim 22 comprising:
c) slurrying a crystalline form of tegaserod maleate in ethanol; and
d) recovering the crystalline tegaserod maleate.

24. The process of claim 23, wherein the crystalline form slurried is characterized by an X-ray Diffraction pattern having peaks at 5.4, 6.0, 6.6 and 10.8±0.2 degrees two theta (Form A).

25. The process of claim 23, further comprising water, methanol, ethyl acetate or mixtures thereof in mixture with the ethanol.

26. The process of claim 23, further comprising heating the slurry, and adding an additional amount of ethanol.

27. A crystalline form of tegaserod maleate (Form B3) characterized by an X-ray Diffraction pattern having peaks at 15.6, 16.0, 22.5, 25.5 and 29.3±0.2 two theta.

28. The crystalline form of claim 27, further characterized by peaks at 7.2, 8.0, 10.3, 16.8, 17.3, 19.6, 20.7, 21.6, 23.3, 24.5, 26.0, 27.2 and 28.0±0.2 two theta.

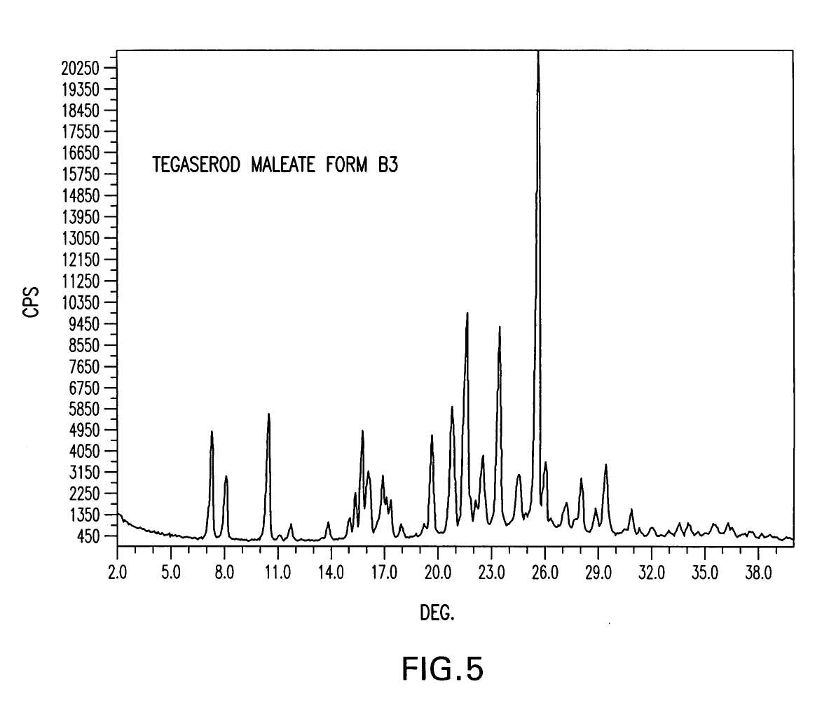

29. The crystalline form of claim 28, wherein the crystalline form has a X-ray diffraction pattern as substantially depicted in FIG. 5.

30. The crystalline form of claim 27, wherein the crystalline form is an ethanol solvate.

31. The crystalline form of claim 27, characterized by a DSC with an endothermic peak at about 140° C., and another endothermic peak at about 185 to about 188° C.

32. The crystalline form of claim 27, wherein the crystalline form has a particle size of below about 250μ and a polymorphic purity of at least about 95% as measured by area percentage XRD.

33. A process for preparing the crystalline form of claim 27 comprising crystallizing the crystalline form from ethanol, or slurrying tegaserod maleate in ethanol or contacting tegaserod maleate with vapors of ethanol.

34. The process of claim 33, wherein at least one of water or methanol are present in addition to ethanol.

35. A process for preparing the crystalline form of claim 27 comprising:
c) combining a solution of maleic acid in ethanol with a solution of tegaserod free base in ethanol; and
d) recovering the crystalline form as a precipitate.

36. A process for preparing crystalline tegaserod maleate characterized by an X-ray Diffraction pattern having peaks at 7.8, 8.7, 17.1, 17.3 and 25.1±0.2 two theta (Form C) comprising heating crystalline tegaserod maleate characterized by an XRD pattern with peaks at 8.7, 15.6, 16.0, 22.2, 25.3 and ±0.2 two theta (Form B2) at a temperature of at least about 40° C.

37. A crystalline form of tegaserod maleate (Form D) having an X-ray powder diffraction with peaks at about 14.6, 20.2, 23.8, 26.0, 28.6 and 29.3±0.2 two theta.

38. The crystalline form of claim 37, further characterized by peaks at 11.1, 17.1, 17.7, 21.6, 22.6, 24.9, 25.2, 27.3, 31.0, 33.9 and 35.8±0.2 two theta.

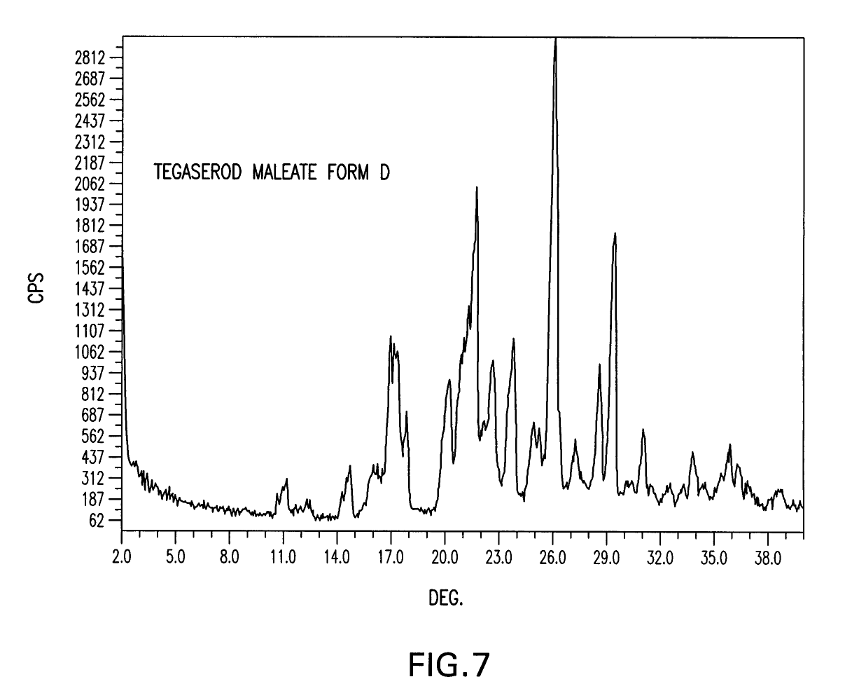

39. The crystalline form of claim 38, wherein the crystalline form has an X-ray difraction pattern as substantially depicted in FIG. 7.

40. The crystalline form of claim 37, wherein the crystalline form has a particle size of below about 250μ and a polymorphic purity of at least about 95% as measured by area percentage XRD.

41. A process for preparing the crystalline form of claim 37, comprising slurrying or crystallizing the crystalline form in a solvent selected from the group consisting of 1-methyl-2-pyrrolidone, n-propanol and mixtures thereof.

42. A crystalline form of tegaserod maleate (Form E) having an X-ray powder diffraction with peaks at 10.3, 16.6, 17.1, 22.0 and 25.4±0.2 two theta.

43. The crystalline form of claim 42, further characterized by peaks at 7.9, 15.9, 19.5, 20.6, 21.4, 22.4, 23.4, 24.4, 26.0, 28.0, 28.5 and 29.3±0.2 two theta.

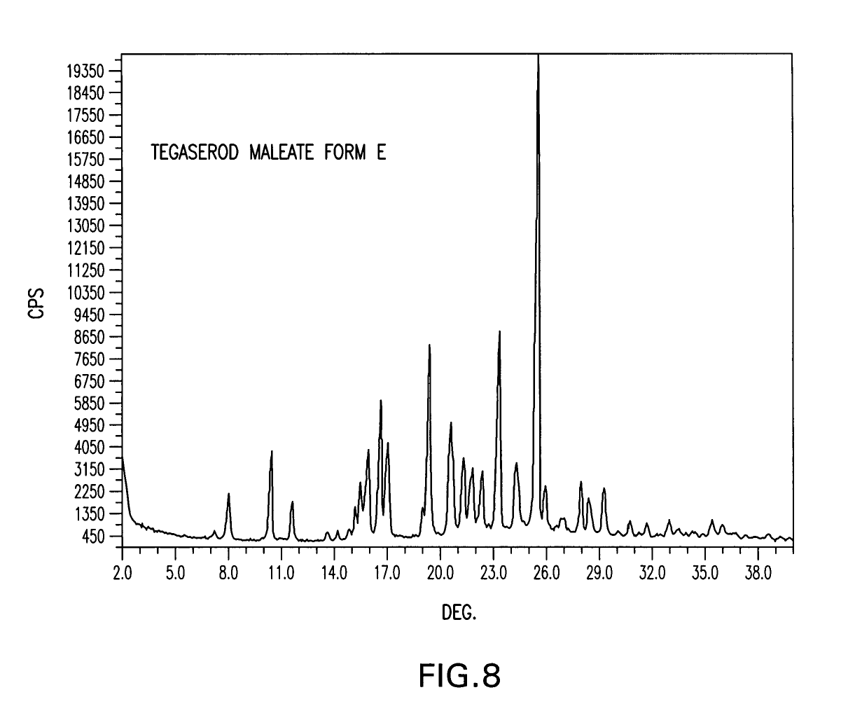

44. The crystalline form of claim 43, wherein the crystalline form has a X-ray diffraction pattern as substantially depicted in FIG. 8.

45. The crystalline form of claim 42, wherein the crystalline form has a particle size of below about 250μ and a polymorphic purity of at least about 95% as measured by area percentage XRD.

46. The crystalline form of claim 42, wherein the crystalline form is a dioxane solvate.

47. The crystalline form of claim 42, characterized by a DSC with an endothermic peak at about 130 EC, and another endothermic peak at about 185 to about 188 EC.

48. A process for preparing the crystalline form of claim 42 comprising:
c) slurrying tegaserod maleate in dioxane; and
d) recovering the crystalline form.

49. The process of claim 48, wherein the tegaserod maleate in step a) is characterized by an X-ray Diffraction pattern having peaks at 5.4, 6.0, 6.6 and 10.8±0.2 degrees two theta (Form A).

50. A process for preparing the crystalline form of claim 42 comprising:
a) combining a solution of maleic acid in tetrahydrofuran with a solution of tegaserod free base in tetrahydrofuran; and
b) recovering the crystalline form as a precipitate.

51. A crystalline form of tegaserod hemi-maleate having an X-ray powder diffraction with peaks at 5.0, 9.9, 19.8, and 25.9±0.2 two theta.

52. The crystalline form of claim 51, wherein the crystalline form is characterized by peaks at 14.2, 14.8, 20.8, 21.5, 23.1 and 23.8±0.2 two theta.

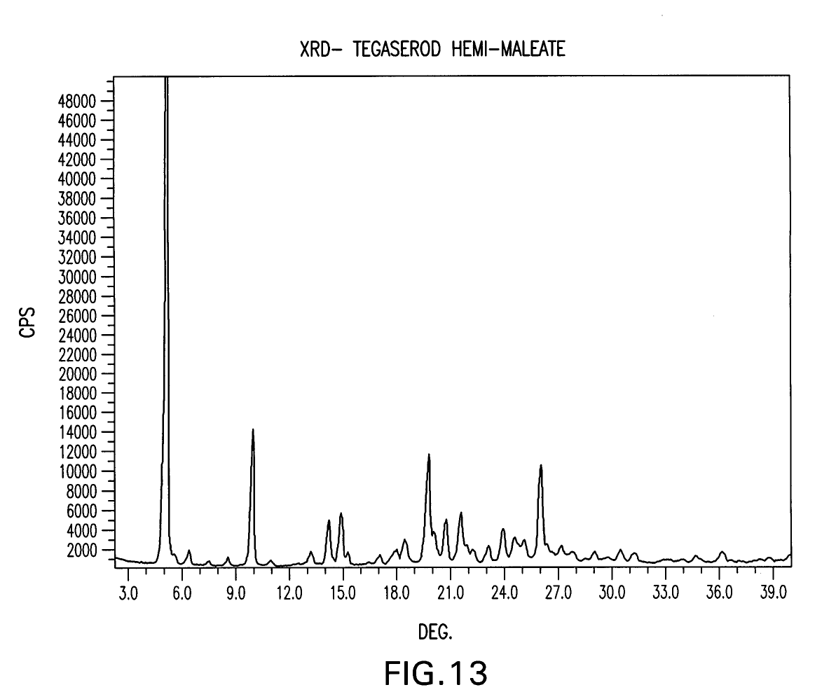

53. The crystalline form of claim 52, wherein the crystalline form has an X-ray diffraction pattern as substantially depicted in FIG. 13.

54. The crystalline form of claim 51, wherein the crystalline form is a hemihydrate.

55. A process for preparing crystalline form of claim 50 comprising:
d) combining tegaserod base, maleic acid and ethyl acetate to obtain a reaction mixture;
e) heating the reaction mixture; and
f) recovering the crystalline form as a precipitate.

56. The process of claim 55, wherein water is added in step a).

57. A crystalline form of tegaserod base (Form F) having an X-ray powder diffraction with peaks at 10.2, 11.3, 20.3, 21.3, 21.8, 27.6, 29.6, 31.1 and 32.7±0.2 two theta.

58. The crystalline form of claim 57, further characterized by peaks at 10.2, 11.3, 15.3, 16.9, 18.3, 19.2, 20.3, 21.3, 21.8, 22.7, 24.4, 27.6, 29.6, 31.1 and 32.7±0.2 two theta.

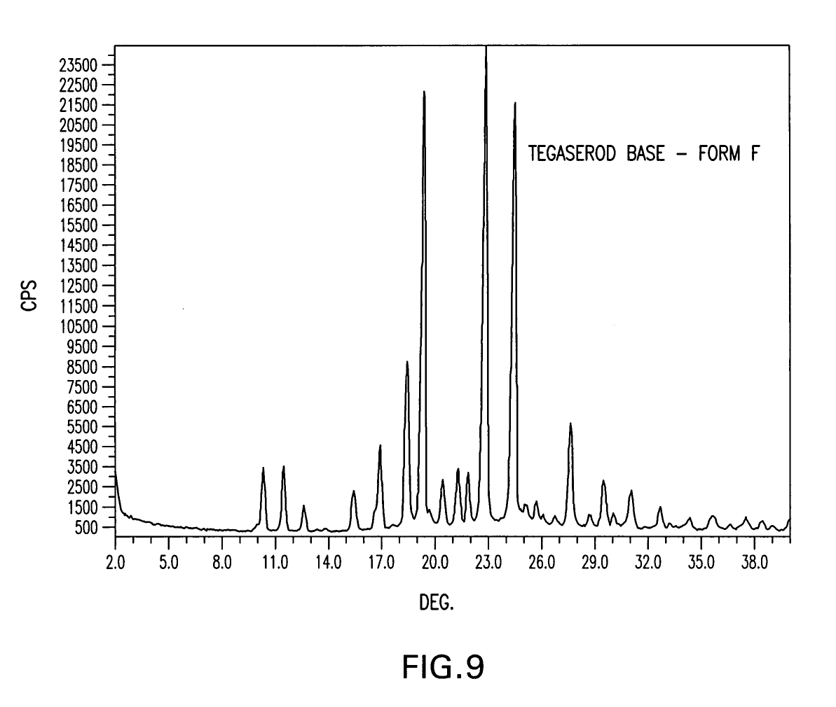

59. The crystalline form of claim 58, wherein the crystalline form has an X-ray diffraction pattern as substantially depicted in FIG. 9.

60. The crystalline form of claim 57, wherein the crystalline form has a particle size of below about 250μ and a polymorphic purity of at least about 95% as measured by area percentage XRD.

61. The crystalline form of claim 57, characterized by a DSC with an endothermic peak at about 154 EC.

62. A process for preparing the crystalline form of claim 57, comprising:
c) preparing a solution of tegaserod in a C₁ to C₈ chlorinated aliphatic hydrocarbon; and
d) removing the chlorinated hydrocarbon.

63. The process of claim 62, wherein the chlorinated hydrocarbon is dichloromethane.

64. The process of claim 62, wherein removing is carried out by evaporation.

65. The process of claim 62, further comprising preliminary steps of distributing tegaserod maleate characterized by an X-ray Diffraction pattern having peaks at 5.4, 6.0, 6.6 and 10.8±0.2 degrees two theta (Form A) between an aqueous phase and the hydrocarbon, contacting the maleate with a base, and recovering the hydrocarbon containing tegaserod.

66. A crystalline form of tegaserod base (Form H) having an X-ray powder diffraction with peaks at 8.8, 15.1, 17.6, 21.8 and 23.9±0.2 two theta.

67. The crystalline form of claim 66, wherein the crystalline form is characterized by peaks at 7.7, 11.9, 16.0, 16.8, 18.1, 19.3, 22.7, 25.4, 26.5 and 29.8±0.2 two theta.

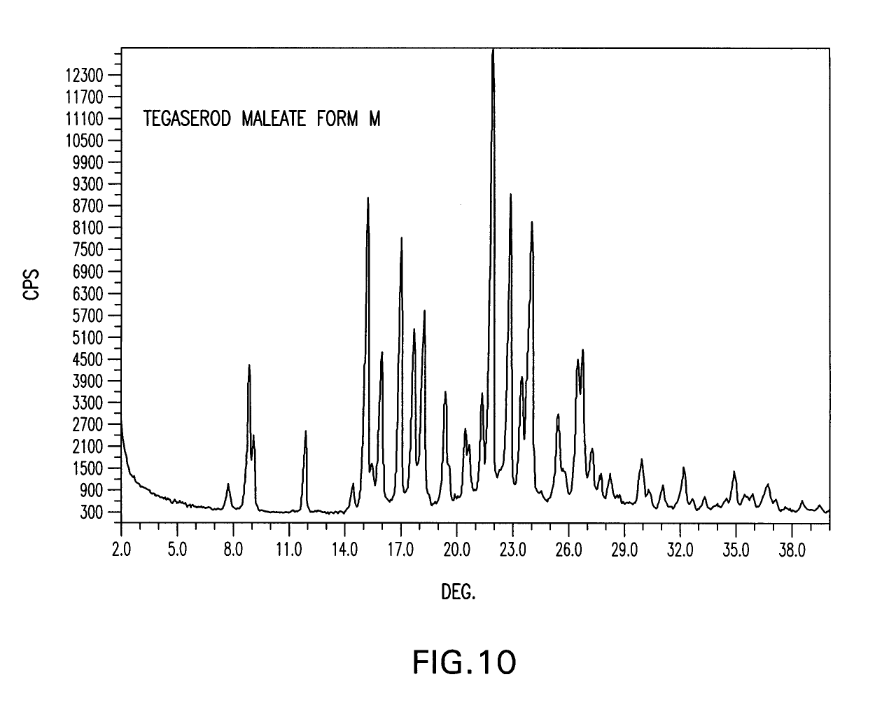

68. The crystalline form of claim 67, wherein the crystalline form has an X-ray diffraction pattern as substantially depicted in FIG. 10.

69. The crystalline form of claim 66, wherein the crystalline form has a particle size of below about 250μ and a polymorphic purity of at least about 95% as measured by area percentage XRD.

70. The crystalline form of claim 66 characterized by a DSC with an endothermic peak at about 134° C., and another endothermic peak at about 156° C.

71. A process for preparing crystalline form of claim 66 comprising:
c) preparing a solution of tegaserod base in ethanol; and
d) recovering the crystalline form as a precipitate.

72. The process of claim 71, wherein precipitation is induced by combining the solution with an anti-solvent.

73. The process of claim 72, wherein the anti-solvent is water.

74. A process for preparing crystalline form of claim 66 comprising:
c) slurrying tegaserod base in ethyl acetate; and
d) recovering the crystalline form from the slurry.

75. Amorphous tegaserod base in the solid state.

76. Amorphous tegaserod of claim 75, wherein the amorphous tegaserod contains less than 10% by weight crystalline tegaserod.

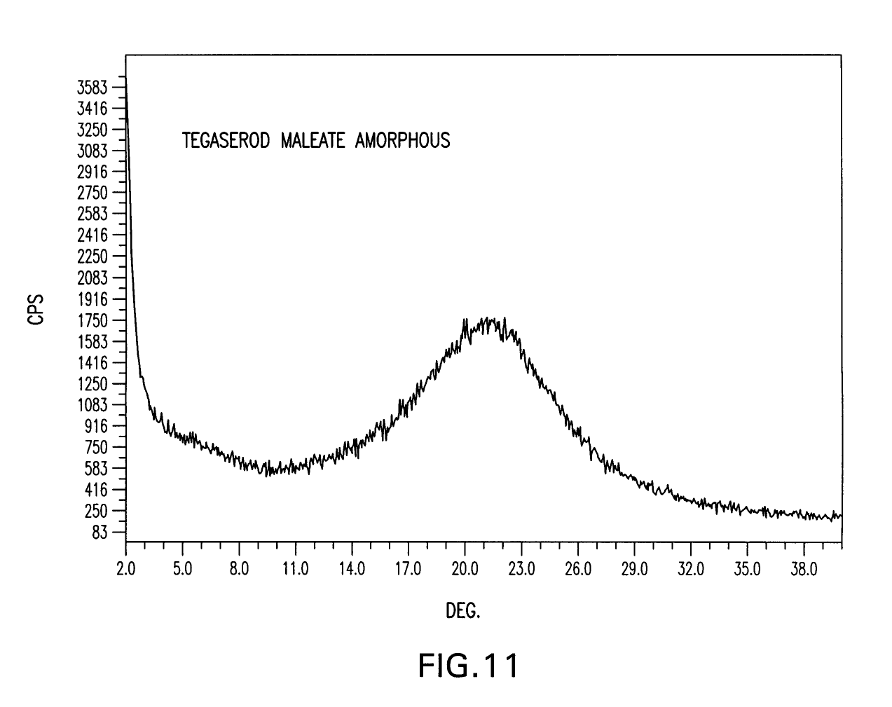

77. The tegaserod of claim 75, wherein the tegaserod has an X-ray diffraction pattern as substantially depicted in FIG. 11.

78. The amorphous tegaserod base of claim 75, characterized by a DSC with an endothermic peak at about 100° C., and other endothermic peaks at about 156° C. and about 132° C.

79. Amorphous tegaserod of claim 75, wherein the amorphous form has a particle size of below about 250μ and contains less than about 95% crystallinity as measured by area percentage XRD.

80. A process for preparing amorphous tegaserod of claim 75 comprising:
c) preparing a solution of tegaserod in an organic solvent; and
d) removing the solvent.

81. The process of claim 80, wherein the organic solvent is a C₁ to C₄ alcohol.

82. The process of claim 80, wherein removing is carried out by evaporation.

83. Tegaserod acetate in solid state.

84. Crystalline tegaserod acetate.

85. Crystalline form of tegaserod acetate (Form J) having an X-ray powder diffraction with peaks at about 7.3, 8.7, 10.9 and 13.5±0.2 two theta.

86. The crystalline form of claim 85, further characterized by peaks at about 18.2, 18.9, 21.8, 23.1 and 24.4±0.2 two theta.

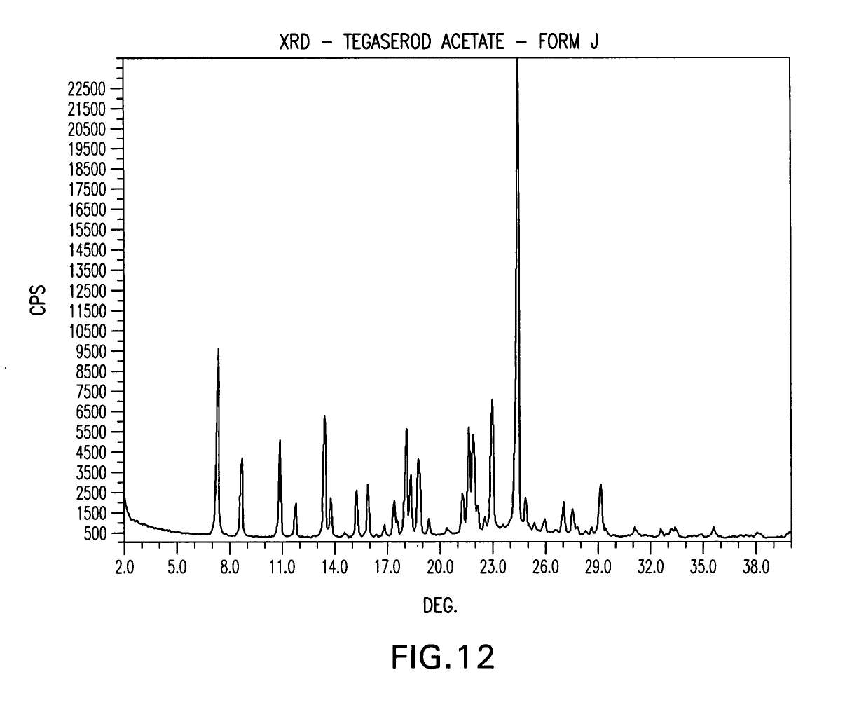

87. The crystalline form of claim 86, wherein the crystalline form has an X-ray diffraction pattern as substantially depicted in FIG. 12.

88. A process for preparing the tegaserod acetate of claim 85 comprising:
c) combining tegaserod salt or base, ethyl acetate or acetic acid, and a base under aqueous condition to obtain a reaction mixture; and
d) recovering the crystalline form.

89. The process of claim 88, wherein the base is sodium hydroxide and the tegaserod salt is tegaserod maleate.

90. A process for preparing the tegaserod acetate of claim 85 comprising:
c) slurrying tegaserod base amorphous in ethyl acetate; and
d) recovering the crystalline form.

91. A pharmaceutical composition comprising a polymorphic form of tegaserod base, maleate or acetate selected from the group consisting of B, B1, B3, D, E, J, tegaserod hemimaleate and a pharmaceutically acceptable excipient.

92. A method of treating a mammal suffering from irritable bowel syndrome comprising administering the pharmaceutical composition of claim 91 to the mammal in need thereof.

93. A solvate of tegaserod maleate, wherein the maleate is a solvate of a solvent selected from the group consisting of ethanol, isopropanol, 1-propanol, chloroform and dioxane.
